(19) United States
(12) Patent Application Publication
(10) Pub. No.: US 2024/0293561 A1
(43) Pub. Date: Sep. 5, 2024

(54) BRAIN PERMEABLE MULTIFUNCTIONAL SYSTEM AND USES THEREOF

(71) Applicant: NANOCARRY THERAPEUTICS LTD., Ness Ziona (IL)

(72) Inventors: Rachela POPOVTZER, Givat Shmuel (IL); Oshra BETZER, Elkana (IL); Yuval SAGIV, SAGIV, Gedera (IL); Revital MANDIL-LEVIN, Ness Ziona (IL); Adam A. ANTEBI, Modiin (IL)

(21) Appl. No.: 18/578,403

(22) PCT Filed: Jul. 13, 2022
(86) PCT No.: PCT/IL2022/050753

§ 371 Date: Jan. 11, 2024

Related U.S. Application Data
(60) Provisional application No. 63/221,498, filed on Jul. 14, 2021.

(51) Int. Cl. A61K 47/60; A61K 9/00; A61K 38/28; A61K 47/69; A61P 25/00

(52) U.S. Cl. CPC A61K 47/60; A61K 9/0085; A61K 38/28; A61K 47/6923; A61K 47/6929; A61P 25/00

(57) ABSTRACT

The present invention provides a BBB-permeable multifunctional system for the synchronized delivery of distinct active agents to the brain. The multifunctional system is based on an inorganic core particle which is conjugated through a first polymeric linker to a first active agent; through a second polymeric linker to a second active agent; and through a third polymeric linker to a brain-internalizing transporter moiety. Further provided are a process for preparation of the multifunctional system, pharmaceutical compositions comprising the multifunctional system and uses thereof in therapeutic and/or diagnostic methods.

### FIELD OF INVENTION

The present invention is in the field of brain-targeted delivery systems for therapeutic and diagnostic uses.

### BACKGROUND OF THE INVENTION

A critical problem in the treatment of brain related diseases, including inter alia neurodegenerative disorders and diseases and brain tumors, is the difficulty in penetrating the blood-brain barrier (BBB) to deliver important therapeutic and diagnostic agents to the brain. The BBB is a highly selective semipermeable border that separates circulating blood from the central nervous system (CNS). The BBB functions mainly as a protective barrier for the brain, preventing transition of various elements, including hormones, neurotransmitters or neurotoxins, from the bloodstream into the CNS. Although specific and selective transporters located on the BBB supply the CNS with glucose, free fatty acids, amino acids, vitamins, minerals, and electrolytes, nearly all high molecular weight (MW) drugs and more than 98% of low molecular weight drugs are unable to cross the BBB. Various strategies are being studied for overcoming the limitations associated with the BBB. Among the many strategies, the utilization of transcytosis trafficking pathways of endogenous receptors expressed at the brain capillary endothelium, represents a promising approach to cross the cellular barrier. Based on this trafficking pathways, various nanomaterial-based drug delivery systems are being developed.

U.S. Pat. No. 10,182,986 is directed to methods of delivering a nanoparticle across the blood brain barrier to the brain of a subject by administering to the subject a nanoparticle having a nanoparticle core and a targeting agent.

Ruan, Shaobo, et al. (Biomaterials 37 (2015): 425-435) provides a gold nanoparticle-based delivery system, which was loaded with doxorubicin (DOX) through hydrazone, an acid-responsive linker, and functionalized with angiopep-2, a specific ligand of low density lipoprotein receptor-related protein-1 (LRP1), which could mediate the system to penetrate blood brain barrier and target to glioma cells.

Shilo, Malka, et al. (Nanoscale 6.4 (2014): 2146-2152) developed a technology, which is directed to transport of insulin-targeted gold nanoparticles (INS-GNPs) through the blood-brain barrier for imaging and therapeutic applications.

Recent progress in cellular biology has led to a paradigm shift in treatment of various challenging diseases, from “one-drug-one-target” approach to combination therapy and multi-targeting drugs approaches. However, although drug combinations can theoretically be therapeutically effective in the treatment of various diseases, their clinical success is limited due to dissimilarities in pharmacokinetics and tissue distribution of each drug in the combination.

Different approaches are being developed to overcome these limitations. Bispecific antibodies (bsAbs) are artificial proteins that combine specificities of two antibodies in a single molecule that simultaneously interferes with multiple surface receptors or ligands. BsAbs can also place targets into close proximity, either to support protein complex formation on one cell, or to trigger contacts between cells.

Recently, nanoparticles have emerged as a promising platform for the co-delivery of multiple drugs. Zhang, Tian, et al. (Advanced healthcare materials 8.18 (2019): 1900543) provides multitargeted nanoparticles that deliver synergistic drugs across the blood-brain barrier to brain metastases of triple negative breast cancer cells and tumor-associated macrophages.

Dixit et al. (Molecular pharmaceutics 12.9 (2015): 3250-3260) disclosed dual receptor-targeted theranostic nanoparticles for localized delivery and activation of photodynamic therapy drug in glioblastomas.

However, there remains an unmet need for multifunctional systems that will enable simultaneous and synchronized delivery of multiple biologically active agents into the brain in order to improve treatment efficacy of brain-related diseases or disorders.

### SUMMARY OF THE INVENTION

The present invention provides a multifunctional system for the co-delivery of at least two distinct active agents into the brain. The present invention further provides methods for preparing the multifunctional system and uses thereof for the treatment of brain-related diseases or disorders.

The multifunctional system of the invention is based on a core particle which is conjugated to a first and a second active agents through a first and a second polymeric linkers, respectively, and to a brain-internalizing transporter moiety, through a third polymeric linker.

The active agents conjugated to the core particle can include various types of therapeutic and/or diagnostic molecules of interest. In particular, it was found that two different antibodies, or an antibody and a small molecule drug, having a poor BBB penetration in their original form, were able to efficiently penetrate into mice brains following intravenous administration, while being conjugated to a single core particle which was further conjugated to insulin as a brain-internalizing transporter moiety.

Moreover, using two different fluorescently labeled antibodies conjugated to the core particle, it was found that the antibodies underwent co-localization within specific brain regions. Therefore, the multifunctional system of the invention not only facilitates the brain penetration of the active agents, but also confers synchronized distribution of the active agents within the brain. Advantageously, synchronized distribution of different therapeutic agents may significantly improve the therapeutic efficacy of the agents' combination.

The present invention is further based in part on the finding that the relative lengths of the first, the second and the third polymeric linkers have a critical impact on the penetration of the multifunctional system through the BBB. In particular, efficient BBB penetration can be achieved when differently sized polymeric linkers are used for conjugating the insulin and the active agents to the core particle.

One of the beneficial features of the system of the present invention is that activity of the therapeutic agents, which are conjugated to the delivery system, remains intact, such that they do not necessarily have to be detached from the nanoparticle after penetration through the BBB, e.g., by using a cleavable linker. Alternatively, the delivery system may comprise different therapeutic agents wherein at least one of them is not cleavable.

According to one aspect, there is provided a multifunctional particle comprising:
(a) an inorganic particle bound to at least: (i) a first linear polymeric linker; (ii) a second linear polymeric linker; and (iii) a third linear polymeric linker;
(b) a first biologically active molecule conjugated to the first linear polymeric linker;
(c) a second biologically active molecule conjugated to the second linear polymeric linker; and
(d) a brain internalizing transporter moiety conjugated to the third linear polymeric linker, wherein the length of the third linear polymeric linker is substantially different than the lengths of the first and the second linear polymeric linkers, wherein the molecular weight (MW) of the third polymeric linker is different than the molecular weight of the first and the second polymeric linkers in at least about 1000 Da, and wherein the first biologically active molecule is distinct from the second biologically active molecule.

According to some embodiments, the length of the third linear polymeric linker is substantially higher than the lengths of the first and the second linear polymeric linkers.

According to some embodiments, the first, the second and the third polymeric linkers are non-cleavable under physiological conditions.

According to some embodiments, the multifunctional particle comprises an additional polymeric linker that is cleavable under physiological conditions. According to some embodiments, the cleavable polymeric linker is conjugated to a chemotherapeutic drug or to a toxin.

According to some embodiments, the molecular weight of the first and the second linear polymeric linkers is within the range of 1,000-10,000 Da and the molecular weight of the third linear polymeric linkers is within the range of 2,000-11,000 Da. In certain embodiments, the molecular weight of the third linear polymeric linker is higher than the molecular weights of the first and the second linear polymeric linkers.

According to some embodiments, the third linear polymeric linker is composed of repeating monomer units and at least one of the first and the second linear polymeric linkers is composed of the same repeating monomer units as the third linear polymeric linker, wherein the third linear polymeric linker has a different number of repeating monomer units than the at least one of the first and the second linear polymeric linkers. In certain embodiments, the first, the second and the third linear polymeric linkers are composed of the same repeating monomer units, wherein the third linear polymeric linker has a different number of repeating monomer units than that of the first and the second linear polymeric linkers.

According to some embodiments, the first polymeric linker and the second polymeric linker are identical.

According to some embodiments, the first and the second linear polymeric linkers are bound to the inorganic particle through a sulfide bond, and the first and the second biologically active molecules are conjugated to the respective linear polymeric linker through an amide bond.

According to some embodiments, the first and the second biologically active molecules are independently selected from the group consisting of an antibody, a peptide, a small molecule, an oligonucleotide, an antisense RNA, and any fragment or combination thereof. In certain embodiments, the first and the second biologically active molecules are both an antibody or a fragment thereof. In other embodiments, the first biologically active molecule is an antibody or a fragment thereof and the second biologically active molecule is a small molecule.

According to some embodiments, the third linear polymeric linker constitutes from about 10% mol to 40% mol of the total polymeric linkers bound to the inorganic particle.

According to some embodiments, each one of the first and the second linear polymeric linkers independently constitutes from about 5% mol to 40% mol of the total polymeric linkers bound to the inorganic particle.

According to some embodiments, the first, the second and the third linear polymeric linkers independently comprise a polymer selected from the group consisting of: a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives, and combinations thereof. According to certain embodiments, at least one of the first, the second and the third linear polymeric linkers is a polyether. In some exemplary embodiments, the polyether is polyethylene glycol (PEG). The polyethylene glycol is selected, according to some embodiments, from a thiolated PEG acid (HS-PEG-COOH), a thiolated PEG amine (HS-PEG-NH₂), and thiolated PEG thiol (SH-PEG-SH), wherein the thiolated end is bound to the inorganic particle and the acid, amine or other thiol end is conjugated to the brain-internalizing transporter moiety or to the respective biologically active molecule. In other embodiments, the polyethylene glycol is SH-PEG-SH, wherein one thiolated end is bound to the inorganic particle and the other thiol end is conjugated to a chemotherapeutic drug or to a toxin. According to some embodiments, the multifunctional particle further comprises a fourth polymeric linker bound to the inorganic particle, wherein the fourth polymeric linker serves a monofunctional purpose in capping terminal functional groups on the particles and to enable distance between the molecules conjugated to the particles, henceforth understood to be interchangeable with the term “cap”. According to some embodiments, said fourth polymeric linker comprises a polymer selected from the group consisting of a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives, and combinations thereof. In some exemplary embodiments, said fourth polymeric linker comprises a polyether, wherein the polyether is methoxy polyethylene glycol (mPEG).

According to some embodiments, the inorganic particle is a nanoparticle selected from the group consisting of a metal nanoparticle, a metal oxide nanoparticle, a ceramic nanoparticle, and any combination thereof. The metal is selected, according to some embodiments, from the group consisting of gold, silver, platinum, iron, and any combination thereof. The metal oxide can be selected from the group consisting of iron oxide, magnesium oxide, nickel oxide, cobalt oxide, aluminum oxide, zinc oxide, copper oxide, manganese oxide, and any combination thereof. In some specific embodiments, the inorganic particle is selected from the group consisting of a gold nanoparticle, an iron(III) oxide nanoparticle, and an iron(II,III) oxide nanoparticle. In further specific embodiments, the inorganic particle is a gold nanoparticle.

According to some embodiments, the inorganic particle is a nanoparticle having a diameter of 10-160 nm.

According to some embodiments, the brain-internalizing transporter moiety is selected from the group consisting of: insulin, an antibody specific for the insulin receptor, transferrin, an antibody specific for the transferrin receptor, a polypeptide that specifically binds to the transferrin receptor, a polypeptide that specifically binds to the insulin receptor, insulin-like growth factor 1 (IGF-1), an antibody specific to IGF-1, a polypeptide that specifically binds to the insulin-like growth factor receptor 1, apolipoprotein Al, B, or E, lactoferrin, angiopep-2, a low-density lipoprotein, an antibody specific for low density lipoprotein receptor or lipoprotein receptor-related protein, a polypeptide that specifically binds to low density lipoprotein receptor or lipoprotein receptor-related protein, an antibody specific for diphtheria toxin receptor, a polypeptide that specifically binds to diphtheria toxin receptor, a BBB-penetrant cell-penetrating peptide (CPP), and any combination thereof. In certain embodiments, the brain-internalizing transporter moiety is insulin or a derivative, analog, conjugate or fragment thereof.

According to some embodiments, the multifunctional particle further comprises a third biologically active molecule, wherein the third biologically active molecule is conjugated to a linear polymeric linker which is bound to the inorganic particle. According to some embodiments, the third biologically active molecule is a chemotherapeutic moiety, or a toxin and it is conjugated to the particle by an SH-PEG-SH linker. According to some embodiments the linker is cleavable and the chemotherapeutic moiety or the toxin is released in the brain.

According to some specific embodiments, the inorganic particle is a gold nanoparticle, the first linear polymeric linker and the second linear polymeric linker are each independently a thiolated PEG3500 acid or thiolated PEG3500 amine, the third linear polymeric linker is a thiolated PEG5000 acid or thiolated PEG5000 amine, the brain-internalizing transporter moiety is insulin and a chemotherapeutic drug is conjugated through a cleavable thiolated PEG3500 thiol linker.

According to some exemplary embodiments, the inorganic particle is a gold nanoparticle, the first linear polymeric linker and the second linear polymeric linker are each independently a thiolated PEG3500 acid or thiolated PEG3500 amine, the third linear polymeric linker is a thiolated PEG5000 acid or thiolated PEG5000 amine, and the brain-internalizing transporter moiety is insulin.

According to some exemplary embodiments, the inorganic particle is a gold nanoparticle, the first linear polymeric linker is a thiolated PEG1000 acid or thiolated PEG1000 amine, the second linear polymeric linker is a thiolated PEG3500 acid or thiolated PEG3500 amine, the third linear polymeric linker is a thiolated PEG5000 acid or thiolated PEG5000 amine, and the brain-internalizing transporter moiety is insulin.

According to another aspect, there is provided a process for preparation of the multifunctional particle according to the various embodiments described hereinabove, the process comprising sequential steps of: (a) partially coating a surface of an inorganic particle with a first linear polymeric linker followed by conjugating the first linear polymeric linker to a first biologically active molecule; (b) partially coating the surface of the inorganic particle with a second linear polymeric linker followed by conjugating said second linear polymeric linker to a second biologically active molecule; and (c) partially coating the surface of the inorganic particle with a third linear polymeric linker followed by conjugating said third linear polymeric linker to a brain internalizing transporter moiety, wherein steps (a), (b) and (c) can be performed in any order.

According to some aspects and embodiments, there is provided a process a for the preparation of a multifunctional particle, the process comprising sequential steps of: (a) partially coating a surface of an inorganic particle with a first linear polymeric linker and a second linear polymeric linker, followed by conjugating the first and the second linear polymeric linkers to a first biologically active molecule and a second biologically active molecule, wherein the first linear polymeric linker and the second linear polymeric linker are identical and wherein the first biologically active molecule is distinct from the second biologically active molecule; and (b) partially coating the surface of the inorganic particle with a third linear polymeric linker followed by conjugating the third linear polymeric linker to a brain internalizing transporter moiety, wherein the length of the third linear polymeric linker is substantially different than the length of the first and the second linear polymeric linkers, wherein the molecular weight of the third polymeric linker is different than the molecular weight of the first and the second polymeric linkers in at least about 1000 Da, and wherein step (a) and step (b) can be performed in any order.

According to some embodiments, the first polymeric linker has a first functional end group configured to bind the first biologically active molecule; the second polymeric linker has a second functional end group configured to bind the second biologically active molecule; and the third polymeric linker has a third functional end group configured to bind the brain internalizing transporter moiety, wherein at least two of the first, the second and the third functional end groups are identical.

According to some embodiments, the process further comprises partially coating the surface of the inorganic particle with a fourth polymeric linker, wherein said fourth polymeric linker is a monofunctional linker used to cap functional groups on the particle and to enable distance between the molecules conjugated to the particles.

According to some embodiments, each one of the first linear polymeric linker and the second linear polymeric linker is added in an amount suitable for covering between 5% and 40% of the surface of the inorganic particle, and the third linear polymeric linker is added in an amount suitable for covering between 5% and 40% of the surface of the inorganic particle.

According to some embodiments, the particle is a gold nanoparticle (GNP) and the process for the preparation of multifunctional gold nanoparticles, comprises the sequential steps of: (a) reduction of HAuCl₄; (b) simultaneous incubation of the reduced GNPs with one mono-functional linker and two different heterofunctional linkers; (c) activation the GNPs to obtain free COOH groups; (d) conjugation of the transporter or other moiety; (d) conjugation of the two different bioactive molecules by incubating with a solution comprising their mixture.

According to some embodiments, the mono-functional linker is mPEG-SH. According to a specific embodiment, the mono-functional linker is mPEG6000-SH or PEG5000-SH and it is added to cover about 80-90% of particle surface.

According to some embodiments, the heterofunctional linker is COOH-PEG-SH. According to some embodiments, one heterofunctional linker is COOH-PEG5000-SH and it is added in a concentration to cover about 15% of particle surface. According to some embodiments, the other heterofunctional linker is COOH-PEG3500-SH and it is added in a concentration to cover about 5% of particle surface.

According to some embodiments, activation of the GNPs is performed by mixing the GNPs with (1-ethyl-3-(3-dimethylaminopropyl) carbodiimide HCl (EDC).

According to some embodiments, the transporter is insulin, and its conjugation is performed by incubating for 1-5 hours with the activated GNPs, at a concentration of about 50-500 IU/ml.

According to some embodiments, the two bioactive molecules are incubated overnight at a concentration of 1-50 mg/ml, with activated GNPs.

Analysis of the GNPs is performed following each step using methods known in the art.

According to some embodiments, the analysis of GNPs is performed using Dynamic Light Scattering (DLS).

According to some embodiments, quantification of the bioactive molecules and the transporter (e.g., insulin) attached to the PEG groups on the GNPs is performed by enzyme-linked immunosorbent assay (ELISA) of the supernatants containing the unbound proteins left after precipitation by centrifugation of the GNPs.

According to yet another aspect, there is provided a pharmaceutical composition comprising the multifunctional particle according to the various embodiments presented hereinabove and a pharmaceutically acceptable carrier, excipient, or diluent.

According to some embodiments, the pharmaceutical composition is for use in the prevention, treatment, and/or monitoring a brain-related disease or disorder in a subject in need thereof.

According to some embodiments, the pharmaceutical composition is for use in simultaneous delivery of at least two biologically active molecules to a brain of a subject.

According to some embodiments, the pharmaceutical composition is formulated for at least one of an intravenous (IV) administration, an intranasal (IN) administration, an intraperitoneal (IP) administration and intrathecal (IT) administration. According to some embodiments, the pharmaceutical composition is for use in the prevention, treatment, and/or monitoring a brain-related disease or disorder in a subject in need thereof.

According to an additional aspect, there is provided a method for a simultaneous delivery of at least two biologically active molecules to a brain of a subject, the method comprising administering to the subject the pharmaceutical composition according to the various embodiments presented hereinabove. According to some embodiments, upon administration, the at least two biologically active molecules exhibit synchronized distribution within the brain.

According to another aspect, there is provided a method of preventing, treating and/or monitoring a brain-related disease or disorder in a subject in need thereof, the method comprising administering to the subject the pharmaceutical composition according to the various embodiments described hereinabove.

According to some embodiments, the pharmaceutical composition is administered to the subject by at least one of an intravenous (IV) administration, an intranasal (IN) administration, an intraperitoneal (IP) administration and intrathecal (IT) administration.

According to some embodiments, the method further comprises a step of imaging the brain of the subject to thereby evaluate accumulation of the multifunctional particle in the brain of said subject. The imaging can be performed using any imaging method or system known in the art including but not limited to an imaging system selected from the group consisting of computed tomography imaging (CT), X-ray imaging, magnetic resonance imaging (MRI), positron emission tomography (PET), single-photon emission computed tomography (SPECT), ultrasound (US), and any combination thereof.

According to some embodiments, the brain-related disease or disorder is a primary brain cancer or secondary brain cancer. According to some embodiments, the brain cancer is a primary solid tumor. According to some embodiments, the brain tumor comprises metastases. According to some embodiments the metastases are derived from a tumor originated in a tissue other than the brain. According to some embodiments, the metastases are originated from a cancer selected from the group consisting of: breast cancer, lung cancer, melanoma, renal cancer and colorectal cancer. According to some embodiments, the metastases are of breast cancer.

According to some embodiments, the brain-related disease or disorder is a primary brain cancer or secondary brain cancer; the inorganic particle is a radiosensitizer; and the method further comprises radiation therapy.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. CL BRIEF DESCRIPTION OF THE DRAWINGS

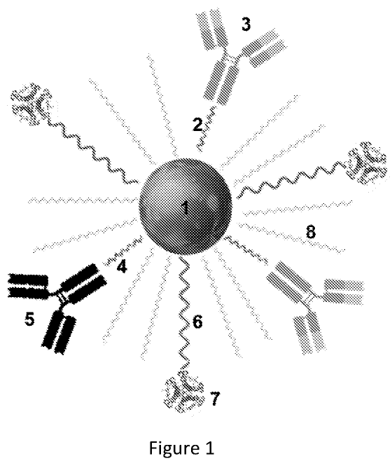

FIG. 1 represents a schematic illustration of a gold nanoparticle (GNP; 1) bound to: (i) a first polymeric linker (2) which is conjugated to a first antibody (3); (ii) a second polymeric linker (4) which is conjugated to a second antibody (5); (iii) a third polymeric linker (6) which is conjugated to insulin (7); and (iv) a monofunctional polymer capping moiety (8).

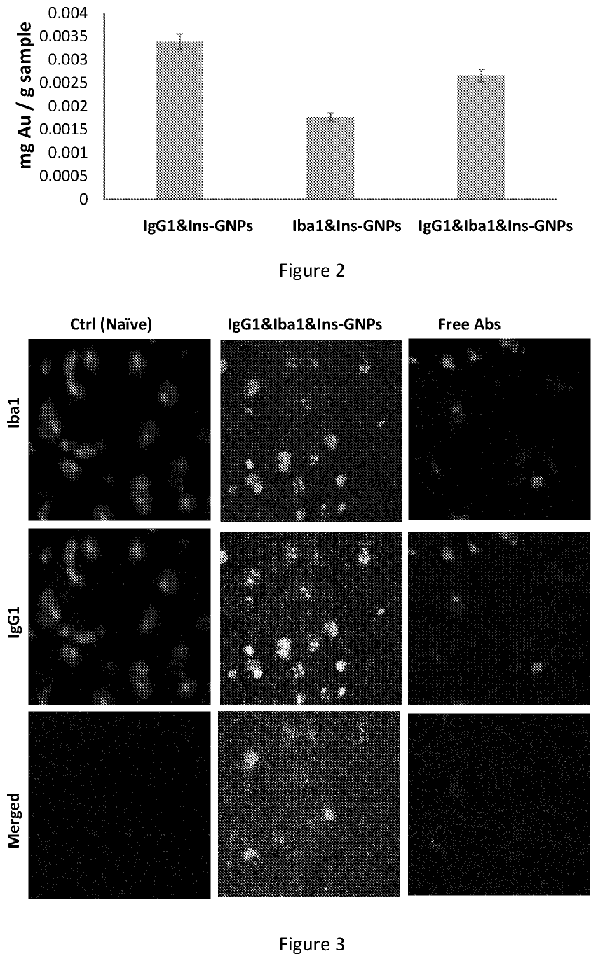

FIG. 2 is a bar graph showing quantification of the amount (mg) of gold (Au) found in brains of mice at 8 hours after intravenous administration of IgG1&Ins-GNPs, Iba1 &Ins-GNPs, and IgG1&Iba1&Ins-GNPs, as measured by inductively coupled plasma-optical emission spectrometry (ICP-OES) analysis.

FIG. 3 represents immunocytochemistry-fluorescence (IHC-F, double staining) images of brain sections of cerebral cortex, obtained from untreated mice (Ctrl, left) or from mice that were intravenously injected with IgG1&Iba1&Ins-GNPs (middle) or with free fluorescently labeled antibodies (right). Upper row images represent Iba1 labeling and 4′,6-diamidino-2-phenylindole (DAPI) staining: Middle row images represent IgG1 labeling and DAPI staining; and bottom row represent merged images of IgG1 and Iba1 labeling (without DAPI labeling).

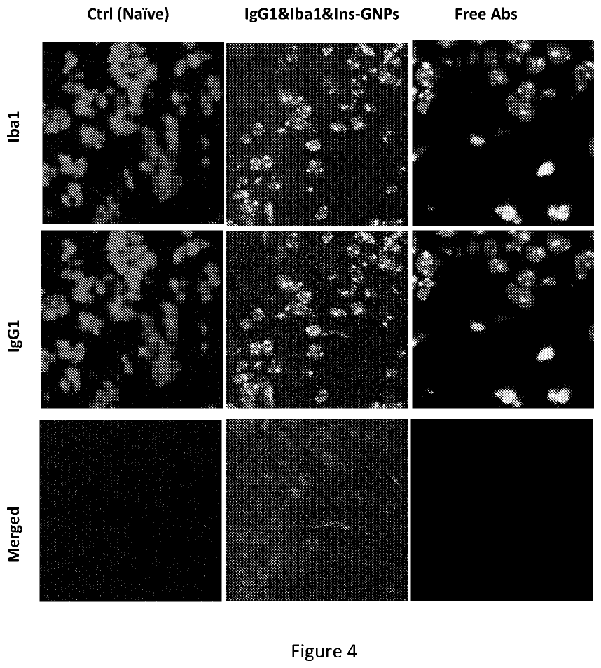

FIG. 4 represents immunocytochemistry-fluorescence (IHC-F, double staining) images of brain sections of the medulla region, obtained from untreated mice (Ctrl, left) or from mice that were intravenously injected with IgG1&Iba1&Ins-GNPs (middle) or with free fluorescently labeled antibodies (right). Upper row images represent Iba1 labeling and DAPI staining: Middle row images represent IgG1 labeling and DAPI staining; and bottom row represent merged images of IgG1 and Iba1 labeling (without DAPI labeling).

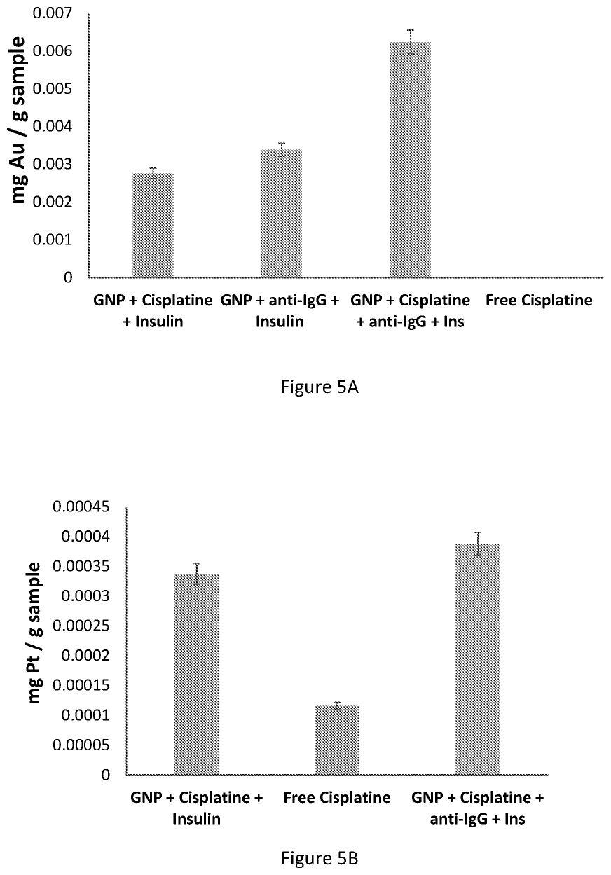

FIG. 5A: Quantification of the amount of Au (mg) found in brain tissue of mice at 8 hours after intravenous administration of cisplatin (cisPt) and insulin (Ins) particles-cisPt&Ins-GNPs, IgG1&Ins-GNPs, cisPt&IgG1&Ins-GNPs or free cisplatin, as measured by ICP-OES analysis.

FIG. 5B: Quantification of the amount of Pt (mg) found in brain tissue of mice at 8 hours after intravenous administration of cisPt&Ins-GNPs, free cisplatin, or cisPt&IgG1&Ins-GNPs, as measured by ICP-OES analysis.

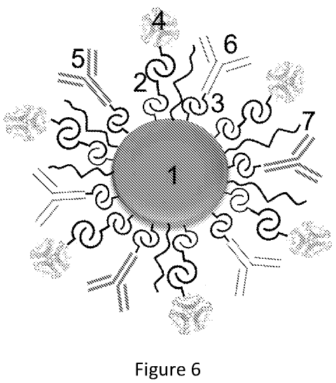

FIG. 6: A schematic illustration of a gold nanoparticle (GNP; 1) bound to: (i) a first polymeric linker (2) which is conjugated to a insulin (4); (ii) a second polymeric linker (3) which is conjugated to a first antibody (5) and to a second antibody (6); and (iii) a capping polymer moiety (7).

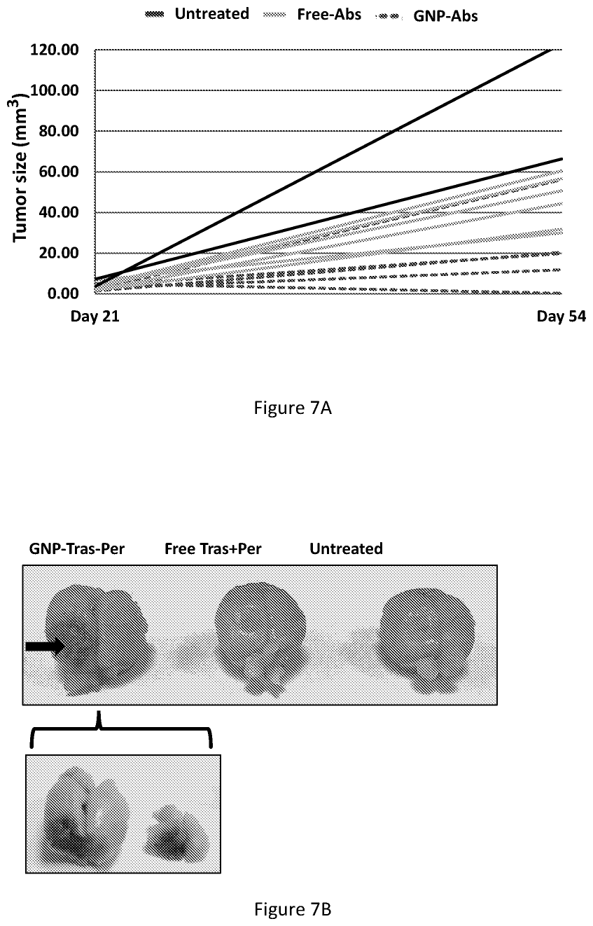

FIG. 7A: Results of an MRI scan of brains of mice inoculated with breast cancer BT474 cells and treated with free antibodies (free-Abs), or bi-functional GNPs (GNP-Abs) or left untreated.

The treatment composition (40 mg/kg Abs) was injected IP once a week, for 4 consecutive weeks.

FIG. 7B: Upper-images of brains extracted and tested by ICP-OES for penetration and tumor accumulation of GNPs. The brains were extracted from mice bearing breast cancer BT474 cells treated with free antibodies (free-Abs), or bi-functional GNPs (GNP-Abs) or left untreated. Lower-cross-section image of the brain of a mouse treated with GNP-Abs.

### DETAILED DESCRIPTION OF THE INVENTION

The present invention provides a generic BBB-permeable platform for the synchronized delivery of distinct active agents to the brain. In particular, the present invention provides a multifunctional system for simultaneous co-delivery of at least two distinct active ingredients into the brain, a process of preparation of the system, pharmaceutical compositions comprising said system, and uses thereof for therapeutic and diagnostic applications.

The multifunctional delivery system is based on a core particle which is conjugated through a first polymeric linker to a first active agent; through a second polymeric linker to a second active agent; and through a third polymeric linker to a brain-internalizing transporter moiety. Each one of the first and the second active agents can be a biologically active molecule (e.g., drug), or a labeling molecule and can comprise different types of molecules such as but not limited to polypeptides, antibodies, peptides, oligonucleotides, and small molecules. Without wishing to be bound by any theory or mechanism, it is hypothesized that the brain-internalizing transporter moiety promotes the penetration of the entire conjugated system through the BBB into the brain. Advantageously, by delivering simultaneously on a single vehicle, two or more distinct active agents having different cellular targets and/or different mechanism of action, an improved therapeutic and/or diagnostic efficacy can be achieved. Thus, the delivery system of the invention can be useful for the treatment and/or diagnosis of a wide range of brain-related diseases or disorders.

The present invention is based in part on the surprising finding that two distinct antibodies having a poor BBB penetration in their original free form, were able to efficiently penetrate into mice brains and to further co-localize within specific brain regions following intravenous administration, while being conjugated to a single core particle which was further conjugated to insulin as a brain-internalizing transporter moiety. It was further shown that the multifunctional system is suitable for the co-delivery of active agents of different types. In particular, it was shown that an antibody and a small molecule drug could be delivered together to mice brains while conjugated to a single core particle.

Any insulin molecule, or an insulin analog, derivative, conjugate or fragment that is capable of binding to endogenous receptors (e.g., insulin receptor) expressed at the human brain capillary endothelium (the BBB), may be used as a transporter according to the present invention. The insulin molecules that may be used according to the present invention include but are not limited to: mammalian insulin, human insulin, recombinant insulin produced by any method know in the art, native and isolated insulin, fast-, rapid- and short-acting insulin and analogs, intermediate-acting insulin and analogs, and long-acting insulin and analogs. Active fragments of any of the above-mentioned insulin molecules may be also used as long as they are capable of binding to endogenous receptors expressed at the human brain capillary endothelium cells and aiding the transport of molecules through the BBB.

According to the principles of the present invention, the first and the second active agents are conjugated to the external surface of the core particle through polymeric linkers, rather than being loaded or encapsulated within the particle core. Importantly, the activity of the active agents is maintained despite this conjugation to the core particle, so that it is not necessarily required to release said agents from the system upon the BBB penetration.

As a diagnostic, this approach enables, in some embodiments, early and accurate detection of brain-related diseases or disorders. For example, when the multifunctional particle comprises one or more biologically active molecules that target the system to diseased or damaged cells within the brain, and the core particle is, or comprises, an imaging agent that enables tracking the particles in vivo using a suitable imaging modality.

As a therapeutic, in some embodiments, this approach enables the delivery of efficient therapeutic agent combinations. In some embodiments, the combination of distinct therapeutic agents on a single platform results in optimized synergism of the agents combination. The different active agents may be targeted to same or different biological entity, such as a receptor, in the cells.

In some embodiments, a combined therapeutic and diagnostic use is enabled, e.g., by using therapeutic active agents that are conjugated to a core particle which constitutes or comprises an imaging contrast agent, e.g., gold nanoparticle.

### Multifunctional System

According to one aspect, there is provided a multifunctional system for the simultaneous-delivery of distinct active agents to the brain, the multifunctional system comprises:
(a) a core particle bound to at least: (i) a first polymeric linker; (ii) a second polymeric linker; and (iii) a third polymeric linker;
(b) a first active agent conjugated to the first polymeric linker;
(c) a second active agent conjugated to the second polymeric linker; and
(d) a brain internalizing transporter moiety conjugated to the third polymeric linker, wherein the first active agent is distinct from the second active agent.

According to another aspect, there is provided a multifunctional system comprising:
(a) a core particle bound to at least: (i) a first polymeric linker; (ii) a second polymeric linker; and (iii) a third polymeric linker; and
(b) a brain internalizing transporter moiety conjugated to the third polymeric linker, wherein each of the first and the second polymeric linkers has a free functional end group configured for conjugating a first active agent and a second active agents, and wherein the first active agent is distinct from the second active agent.

In some embodiments, the length of the third polymeric linker is substantially different than the length of at least one of the first and the second polymeric linkers. In some embodiments, the length of the third polymeric linker is substantially higher than the length of at least one of the first and the second polymeric linkers.

In some embodiments, the first and the second active agents are independently selected from a biologically active molecule and a labeling molecule.

The term “multifunctional system”, which can be used herein interchangeably with the terms “multifunctional particle” and “co-delivery system”, refers to a system that is capable of accomplishing at least two objectives or is capable of performing a single advanced function through incorporation of at least two functional units. The system of the invention incorporates multiple functional units having distinct objectives, including at least the first and the second active agents which have distinct targets and/or distinct activity, and the brain internalizing transporter moiety which acts as a molecular Trojan horse to deliver the system across the BBB.

As used herein, the term “co-delivery” can be interchangeably used with the term “simultaneous delivery” and means that the two distinct active agents are delivered simultaneously in a single composition to their target, e.g., to the brain of a subject or to specific region in the brain of a subject. In some embodiment, “co-delivery” means synchronized delivery, i.e., that upon administration, the distinct active agents exhibit synchronized pharmacokinetics and biodistribution. In some related embodiments, the two active agents exhibit synchronized distribution within the brain. The term “synchronized distribution” as used herein means that the two active agents co-localize within the same brain regions/cells. In some embodiments, the two active agents accumulate in the same brain region/s.

In some embodiments, in particular when the first and the second active agents are both therapeutic agents, the synchronized pharmacokinetics and biodistribution results in synergism of the agents combination and improvement in the therapeutic response.

The terms “delivery” and “delivered” encompass both delivery of the active agent(s) by releasing said active agent(s) from the delivery system (e.g., by using cleavable linkers), and delivery of the active agent(s) while being conjugated (e.g., by covalent conjugation) to the delivery system. Advantageously, the composition of the multifunctional system of the invention does not interfere with the functionality of the active agents, such that releasing the active agents from the system is not necessarily required. According to some embodiments, the multifunctional system comprises non-cleavable first and second linkers connecting active molecules and a cleavable linker connecting a chemotherapeutic drug or a toxin.

The term “distinct” as used herein means that the first active agent molecule is distinguishably different than the second active agent molecule. It is to be understood that the term “distinct” encompass also different molecules of the same type, e.g., two antibodies having different specificities, and two different molecules targeted to same or distinct biological entity. It is further to be understood that the term “distinct” also encompass different molecules that comprise a similar specificity. For Example, a whole antibody (e.g., IgG) and a fragment of said antibody (e.g., Fc/Fab region) are considered as distinct active agents.

As used herein, the term “core particle” refer to a particle which constitutes the central part of the co-delivery system. In some embodiments, the core particle is a nanoparticle. The term “nanoparticle” refers to a particle having a diameter of between 1 to 1000 nm.

In some embodiments, the core particle is selected from the group consisting of a metal particle, a metal oxide particle, a metal carbide particle, a lipid particle, a carbon-based particle, a ceramic particle, a polymeric particle and a liposome. Each possibility represents a separate embodiment of the present invention. In some embodiments, the core particle is an inorganic particle. In some embodiments, the inorganic particle is selected from the group consisting of a metal particle, a metal oxide particle and a ceramic particle. In some embodiments, the inorganic particle is selected from the group consisting of a metal particle and a metal oxide particle. In some embodiments, the inorganic particle is metal particle. In other embodiments, the inorganic particle is a metal oxide particle. In specific embodiments, the inorganic particle is selected from a gold particle and an iron oxide particle.

In some embodiments, the metal particle is a magnetic particle. In some embodiments, the inorganic particle is a magnetic particle. In some embodiments, the magnetic particle is a contrast agent for magnetic resonance imaging (MRI). Any magnetic particle suitable for use as an MRI contrast agent may be used in the composition and methods of the present invention. The magnetic particle may be formed, at least in part, from any material affected by a magnetic field. Examples of suitable materials include, but are not limited to magnetite, hematite, ferrites, and materials comprising one or more of iron, cobalt, manganese, nickel, chromium, gadolinium, neodymium, dysprosium, samarium, erbium, iron carbide, iron, or a combination thereof.

In some embodiments, the inorganic particle is a contrast agent for computed tomography (CT) or X-ray imaging. In some embodiments, the inorganic particle is a metal particle which can be used as a CT or X-ray imaging contrast agent. As will be apparent to those skilled in the art, any metal and/or combination of metals suitable for use for imaging by CT or X-ray may be used in the metal particle of the present invention, in embodiments related to diagnostic use. In some embodiments, metals which can be used to form the particle of the invention are heavy metals, or metal with a high Z number. Examples of suitable metals include, but are not limited to: gold, silver, platinum, palladium, cobalt, iron, copper, tin, tantalum, vanadium, molybdenum, tungsten, osmium, iridium, rhenium, hafnium, thallium, lead, bismuth, gadolinium, dysprosium, holmium, and uranium, or a combination thereof.

In some embodiments, the multifunctional particle consists essentially of:
(a) an inorganic particle bound to: (i) a first linear polymeric linker; (ii) a second linear polymeric linker; and (iii) a third linear polymeric linker;
(b) a first biologically active molecule conjugated to the first linear polymeric linker;
(c) a second biologically active molecule conjugated to the second linear polymeric linker; and
(d) a brain internalizing transporter moiety conjugated to the third linear polymeric linker, wherein the length of the third linear polymeric linker is substantially different than the lengths of the first and the second linear polymeric linkers, wherein the first biologically active molecule is distinct from the second biologically active molecule, and wherein the inorganic particle is an imaging agent that can be detected by an imaging modality selected from computed tomography imaging (CT), X-ray imaging, magnetic resonance imaging (MRI), positron emission tomography (PET), single-photon emission computed tomography (SPECT), ultrasound (US), and any combination thereof. Advantageously, in such embodiments, the multifunctional particle can be used in diagnostic applications without the need to conjugate a labeling molecule as an active agent.

According to some embodiments, the inorganic particle is a metal particle selected from the group consisting of a gold particle, a silver particle, a platinum particle, an iron particle, a copper particle, and a mixture or combination thereof. Each possibility represents a separate embodiment. In some embodiments, the metal particle is a gold (Au) particle.

In some embodiments, the inorganic particle is a metal oxide particle. In some embodiment, the metal oxide particle is selected from the group consisting of iron oxide (Fe₂O₃ or Fe₃O₄), magnesium oxide, nickel oxide, cobalt oxide, aluminum oxide, zinc oxide, copper oxide and manganese oxide, or any combination thereof. Each possibility represents a separate embodiment of the present invention. In some embodiment, the metal oxide particle comprises iron oxide selected from iron(III) oxide and iron(II,III) oxide. In some embodiments, the metal oxide particle is an iron oxide particle wherein the iron oxide is selected from iron(III) oxide and iron(II,III) oxide.

In some embodiments, the core particle is selected from the group consisting of a lipid particle, a carbon-based particle, a ceramic particle, a polymeric particle and a liposome.

In some embodiments, the core particle is a radiosensitizer. The term “radiosensitizer” as used herein refers to an agent that makes cells (particularly cancer cells) more sensitive to radiation therapy. Typically, materials having high atomic number, such as gold (Z=79) increase radiation sensitivity. Accordingly, a gold nanoparticle is an example of a core particle which is a radiosensitizer.

According to some embodiments, the core particle is a nanoparticle having a diameter of 1-200 nm, 1-180 nm, 1-160 nm, 1-140 nm, 1-120 nm, 1-100 nm, 1-90 nm, 1-80 nm, 1-70 nm, 1-60 nm, 1-50 nm, 1-40 nm, 2-100 nm, 2-60 nm, 2-50 nm, 2-40 nm, 2-30 nm, 2-20 nm, 2-10 nm, 3-100 nm, 3-60 nm, 3-50 nm, 3-40 nm, 3-30 nm, 3-20 nm, 4-100 nm, 4-60 nm, 4-50 nm, 4-40 nm, 5-200 nm, 6-190 nm, 7-180 nm, 8-170 nm, 10-160 nm, 20-160 nm, 10-150 nm, 10-140 nm, 10-120 nm, 10-110 nm, 10-100 nm, 10-90 nm, 10-80 nm, 12-70 nm, 14-60 nm, 15-50 nm, 15-40 nm, 15-30 nm, 20-30 nm, 15-30 nm, 20-90 nm, 20-80 nm, 20-70 nm, 20-60 m, 20-50 nm, 20-40 nm, 20-30 nm, 30-70 nm, 30-60 nm, 40-60 nm, 10-200 nm, 20-200 nm, 30-200 nm, 40-200 nm, 50-200 nm, 60-200 nm, 70-200 nm, 80-200 nm 90-200 nm, 100-200 nm, 110-190 nm, 120-170 nm, 130-160 nm, 100-160 nm, 80-160 nm, 60-160 nm, 40-160 nm, 20-160 nm, 10-160 nm, 20-150 nm or 30-150 nm. Each possibility represents a separate embodiment of the present invention. According to some embodiments, the core particle is nanoparticle having a diameter of at least 1 nm, at least 2 nm, at least 3 nm, at least 4 nm, at least 5 nm, at least 10 nm, at least 12 nm, at least 15 nm, at least 18 nm, at least 20 nm, at least 25 nm, at least 30 nm, at least 35 nm, at least 40 nm, at least 45 nm, at least 50 nm, at least 60 nm, at least 70 nm, at least 80 nm, at least 90 nm, at least 100 nm, at least 110 nm, at least 120 nm, at least 130 nm, at least 140 nm or at least 150 nm. Each possibility represents a separate embodiment of the present invention. According to some embodiments, the core particle is a nanoparticle having a diameter of at most 5 nm, at most 10 nm, at most 15 nm, at most 20 nm, at most 30 nm, at most 40 nm, at most 50 nm, at most 60 nm, at most 70 nm, at most 80 nm, at most 90 nm, at most 100 nm, at most 120 nm, at most 140 nm, at most 160 nm, at most 180 nm or at most 200 nm. Each possibility represents a separate embodiment of the present invention.

According to some embodiments, the multifunctional particle, i.e., the entire co-delivery system has a diameter of 5-500 nm, 6-400 nm, 8-300 nm, 10-300 nm, 10-200 nm, 10-180 nm, 10-160 nm, 10-150 nm, 10-100 nm, 20-90 nm, 20-80 nm, 20-70 nm, 20-60 nm, 25-100 nm, 25-90 nm, 25-80 nm, 25-70 nm, 25-60 nm, 25-50 nm, 30-60 nm, 40-200 nm, 40-150 nm, 40-120 nm, 40-100 nm, 40-80 nm, 40-60 nm, 50-300 nm, 50-250 nm, 50-200 nm, 50-180 nm, 50-150 nm, 60-200 nm, 70-180 nm, 80-180 nm, 90-170 nm, 100-160 nm, 100-200 nm, 150-200 nm or 150-180 nm. According to some embodiments, the multifunctional particle has a diameter of 2-200 nm, 1-100 nm, 1-150 nm, 1-200 nm, 2-50 nm, 2-100 nm, 2-150 nm, 4-50 nm, 4-100 nm, 4-150 nm, or 4-200 nm. Each possibility represents a separate embodiment of the present invention. According to some embodiments, the multifunctional particle has a diameter of at least 1 nm, at least 2 nm, at least 5 nm, at least 10 nm, at least 15 nm, at least 20 nm, at least 25 nm, at least 30 nm, at least 35 nm, at least 40 nm, at least 45 nm, at least 50 nm, at least 55 nm, at least 60 nm, at least 70 nm, at least 80 nm, at least 90 nm, at least 100 nm, at least 110 nm, at least 120 nm, at least 130 nm, at least 140 nm, at least 150 nm, at least 160 nm, at least 180 nm, or at least 200 nm. Each possibility represents a separate embodiment of the present invention. According to some embodiments, the multifunctional particle has a diameter of at most 5 nm, at most 20 nm, at most 30 nm, at most 40 nm, at most 50 nm, at most 60 nm, at most 70 nm, at most 80 nm, at most 90 nm, at most 100 nm, at most 110 nm, at most 120 nm, at most 130 nm, at most 140 nm, at most 150 nm, at most 180 nm, at most 200 nm, at most 250 nm, at most 300 nm, at most 350 nm, at most 400 nm, at most 450 nm or at most 500 nm. Each possibility represents a separate embodiment of the present invention.

As used herein, the term “diameter” of a particle/nanoparticle can be used interchangeably with the term “size” of a particle/nanoparticle and refers to the largest linear distance between two points on the surface of a described particle/nanoparticle. The term “diameter”, as used herein, encompasses sizes of spherical particles as well as of non-spherical particles, and may refer to the actual size of the particle or to its hydrodynamic diameter that includes contributions from the solvation sphere. Any method known in the art can be used to determine the size of the particle, for example transmission electron microscopy (TEM), scanning electron microscopy (SEM), and dynamic light scattering (DLS). The term “diameter” may refer to a mean diameter of a plurality of particles measured by any of the above-mentioned techniques.

The core particle is coated with a polymeric layer comprising at least three polymers: a first polymeric linker which has a functional end group which is capable of binding a first active agent, a second polymeric linker which has a functional end group which is capable of binding a second active agent, and a third polymeric linker conjugated to a brain-internalizing transporter moiety. In some embodiments, the first polymeric linker is conjugated to the first active agent.

In some embodiments, the second polymeric linker is conjugated to the second active agent. In some embodiments, the core particle comprises an additional polymeric linker which has a functional end group which is capable of binding a chemotherapeutic drug or a toxin. According to some embodiments, said additional polymeric linker is cleavable. According to some embodiments, said cleavable polymeric linker is an SH-PEG-SH linker.

The term “coated” as used herein is intended to mean that a layer, e.g., a polymeric layer comprising a plurality of polymer moieties, is chemically attached to the surface of the core particle and thereby at least partly covers said core particle. A “particle coated with a polymeric layer” means that each polymer moiety in the polymeric layer is chemically attached to the particle through a functional end group, e.g., a thiol group, of said polymer moiety. The chemical attachment can be covalent, semi-covalent or non-covalent.

The term “polymer moiety” can be interchangeably used with the term “polymer” and refers to a molecule that contains two or more repeating subunits linked in a linear, branched, hyperbranched, dendritic or cyclic sequence, or any combination thereof. In some embodiments, the term “polymer moiety” refers to a molecule that contains at least 3 repeating subunits linked in a linear, branched, hyperbranched, dendritic or cyclic sequence, or any combination thereof. Examples of subunits include alkylene, arylene, heteroalkylene, amino acid, nucleic acid, saccharide, and the like. Examples of polymer moieties include but are not limited to poly (ethylene glycol) groups, poly (ethylene amine) groups, and poly (amino acid) groups. The terms “polymer moiety” and “polymer” encompass also polymeric linkers. As used herein, the term “polymeric linker” refers to a polymer moiety, which originally comprises at least one functional/reactive group that enables binding to a substance, e.g., a particle. In some embodiments, polymeric linker is a bifunctional polymer having at least two functional/reactive groups that enable binding to at least two substances thereby linking between said at least two substances. In some embodiments, polymeric linker is a monofunctional polymer having one functional/reactive group that enables binding to one substance, e.g., a core particle. It should be understood that the terms “monofunctional”, “bifunctional”, “functional group”, etc., as used herein, relate to the polymeric linker according to its original form prior to attachment to the core particle and/or to the brain internalizing transporter moiety or to the respective active agent.

In some embodiments, the core particle is bound to a first polymeric linker. In some embodiments, the core particle is bound to a second polymeric linker. In some embodiments, the core particle is bound to a third polymeric linker. In some embodiments, the core particle is bound to a first, a second and a third polymeric linkers.

The term “bound” can be interchangeably used with the term “conjugated”. In some embodiments, bound is covalently conjugated. The terms “covalent attachment”, “covalently attached”, “covalently linked” and “covalently bonded” are used herein interchangeably, and refer to the forming of a chemical bonding that is characterized by the sharing of pairs of electrons between atoms. For example, a covalently attached agent coating refers to an agent coating that forms chemical bonds with a functionalized surface of a substrate, as compared to attachment to the surface via other means, for example, adhesion or electrostatic interaction. It will be appreciated that agents (e.g., polymers) attached covalently to a surface can also be bonded via other means in addition to covalent attachment.

In some embodiments, the polymer moieties and/or linkers are attached to the external surface of the core particle via a chemical attachment selected from the group consisting of: covalent attachment, semi-covalent attachment and non-covalent attachment. Each possibility represents a separate embodiment of the present invention. In some embodiments, the polymer moieties and/or linkers are attached to the external surface of the core particle via a semi-covalent attachment. As used herein, the term “semi-covalent attachment” refers to a coordinate bond wherein the shared pair of electrons which form the bond come from the same atom. In the present disclosure, a semi-covalent attachment may occur between a metal particle, e.g., gold particle, and thiol groups.

In some embodiments, at least one of the first, the second and the third polymeric linkers is a linear polymeric linker. In some embodiments, the first polymeric linker is a linear polymeric linker. In some embodiments, the second polymeric linker is a linear polymeric linker. In some embodiments, the third polymeric linker is a linear polymeric linker. In some embodiments, the first and the second polymeric linkers are linear polymeric linkers. In some embodiments, the first and the third polymeric linkers are linear polymeric linkers. In some embodiments, the second and the third polymeric linkers are linear polymeric linkers. In some embodiments, the first, the second and the third polymeric linkers are linear polymeric linkers. In some embodiments, the linear polymeric linker is a bifunctional linear polymer having two functional/reactive groups on the two ends of said linear polymer. In some embodiments, each one of the first, the second and the third polymeric linkers is independently a linear bifunctional polymeric linker having two functional/reactive groups on the two ends of said linear polymer.

As used herein, the term “linear” polymer/polymeric linker refers, in some embodiments, to a polymer/polymeric linker in which at least 80% of monomer units are connected in a linear fashion, i.e., in the form of a single-strand polymer chain. In further embodiments, the term “linear” polymer/polymeric linker refers to a polymer/polymeric linker in which at least 90% of monomer units are connected in a linear fashion. In yet further embodiments, the term “linear” polymer/polymeric linker refers to a polymer/polymeric linker in which about 100% of monomer units are connected in a linear fashion. The term “single-strand polymer chain” as used herein, refers to a polymer chain that comprises monomers connected in such a way that monomer units are joined to each other through two atoms, one on each monomer unit.

In some embodiments, the multifunctional system further comprises an additional polymer moiety bound to the core particle. In some embodiments, the additional polymer moiety is a linear polymer. In some embodiments, the additional polymer moiety is a monofunctional polymer used as capping to close or inactivate functional groups on the particles and to enable distance between linkers carrying the bioactive molecules. In some embodiments, the additional polymer moiety is a monofunctional polymeric linker. The additional polymer moiety is thus, in some embodiments, a fourth polymeric linker bound to the core particle. In some embodiments, the core particle is bound to a first, second, third and fourth polymeric linkers. In some embodiments, the fourth polymeric linker is monofunctional, i.e., originally having a single functional end group configured for conjugating said polymeric linker to the core particle and to be used as a capping moiety. In some embodiments, the fourth polymeric linker is a linear monofunctional polymer.

In some embodiments, the first polymeric linker comprises a polymer selected from the group consisting of, but not limited to a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives, and combinations thereof. Each possibility represents a separate embodiment of the invention.

The term “derivative” as used herein refers to a compound whose core structure is the same as, or closely resembles that of, a parent compound, but which has a chemical or physical modification, such as a different or additional group, such as, but not limited to, an alkoxy group, a carboxy group, an amine group, a methoxy group and a thiol group.

In some embodiments, the first polymeric linker comprises a polyether. In some embodiments, the first polymeric linker is a polyether. In some embodiments, the polyether is polyethylene glycol (PEG) or a derivative thereof.

Where appropriate, the abbreviation (PEG) is used in combination with a numeric suffix which indicates the average molecular weight of the PEG. A form of PEG or a PEG species is a PEG or PEG derivative with a specified average molecular weight.

As used herein “PEG or derivatives thereof” refers to any compound including at least one polyethylene glycol moiety. PEGs exist in linear forms and branched forms comprising a multi-arm and/or grafted polyethylene glycols. The term “PEG derivative”, as used herein, relates to PEG which is modified by alkylation of the terminal hydroxy group. In some embodiments, the terminal hydroxyl group is alkylated by a linear or branched C1-C6 alkyl. A PEG may further comprise a functional group. A PEG may be mono-, di-, or multifunctional polyethylene glycols.

Exemplary functional groups include, but are not limited to, the following: a hydroxyl, a carboxyl, a thiol, an amine, a phosphate, a phosphonate, a sulfate, a sulfite, a sulfonate, a sulfoxide, a sulfone, an amide, an ester, a ketone, an aldehyde, a cyano, an alkyne, an azide, and an alkene, or a combination thereof.

In some embodiments, the first polymeric linker comprises a thiol (—SH) end group. In some embodiments, said first polymeric linker is chemically attached to the core particle through said thiol (—SH) end group. In some embodiments, the first polymeric linker is conjugated to the first active agent through an amide bond. In some embodiments, the core particle is bound to the first polymeric linker through a sulfide bond and the first active agent is conjugated to said first polymeric linker through an amide bond. In some embodiments, the core particle is an inorganic particle and is bound to the first polymeric linker through a sulfide bond and the first active agent is conjugated to said first polymeric linker through an amide bond. In some embodiments, the first polymeric linker within the co-delivery system has a structure —S—R—CONH—, wherein R is a polymeric chain consisting of repeating monomer units. In other embodiments, the first polymeric linker within the co-delivery system has a structure —S—R—NHCO—, wherein R is a polymeric chain consisting of repeating monomer units. In some embodiments, the first polymeric linker is selected from thiolated PEG acid (HS-PEG-COOH) and thiolated PEG amine (HS-PEG-NH₂). It is to be understood that the HS and COOH/NH₂ end groups refer to the polymeric linker prior to conjugation with the core particle and the active agent. In some embodiments, the thiol group is chemically attached to the core particle and the acid or amine group is covalently conjugated to the first active agent. In some embodiments, the first polymeric linker within the co-delivery system has a structure selected from —S-PEG-C(O)— and —S-PEG-NH—.

In some embodiments, the first polymeric linker is a non-cleavable linker. In some embodiments, the first polymeric linker is non-cleavable under physiological conditions.

The term “non-cleavable” as used herein refers to a stable bond that is not acid or base sensitive, not sensitive to reducing or oxidizing agents, and not sensitive to enzymes that can be found in cells or the circulatory system. In some embodiments, non-cleavable polymeric linkers are devoid of pH sensitive hydrazones. In some embodiments, non-cleavable polymeric linkers are devoid of disulfide bonds. In some embodiments, non-cleavable polymeric linkers are devoid of ester bonds. It is to be understood that the term “polymeric linker is non-cleavable”, is meant to encompass the bond between the core particle and the polymeric linker; the bond between the respective polymeric linker and the respective active agent; or the bond between the respective polymeric linker and the brain internalizing transporter moiety, as well as any bond within the polymeric linker itself.

In yet some embodiments, the particle comprises a chemotherapeutic drug or a toxin connected through a cleavable linker, e.g., an SH-PEG-SH linker.

In some embodiments, the first polymeric linker has a molecular weight (MW) between 2,000 to 7,000 Dalton (Da). In some embodiments, the first polymeric linker has a molecular weight (MW) within a range selected from the group consisting of 500-10,000 Da, 1,000-10,000 Da, 600-9,500 Da, 700-9,000 Da, 800-8,500 Da, 800-6,000 Da, 800-5,000 Da, 800-4,000 Da, 800-3,000 Da, 800-2,000 Da, 900-8,000 Da, 1,000-7,000 Da, 1,500-6,500 Da, 2,000-6,000 Da, 3,000-6,000 Da, 4,000-6,000 Da, 1,000-2,000 Da, 1,000-3,000 Da, 1,000-4,000 Da, 1,000-5,000 Da, 1,000-7,000 Da, 3,400-7,000 Da, 2,000-3,000 Da, 2,000-5,000 Da, 2,000-7,000 Da, 2,000-10,000 Da, 3,000-3,400 Da, 3,000-4,000 Da 3,000-5,000 Da, 3,000-7,000 Da, 3,000-10,000 Da, 5,000-7,000 Da, 5,000-10,000 Da, and 7,000-10,000 Da. Each possibility represents a separate embodiment. According to some embodiments, the first polymeric linker has a MW of at least 1,000 Da, at least 1,500 Da, at least 2,000 Da, at least 2,500 Da, at least 3,000 Da, at least 3,400 Da, at least 4,000 Da, at least 5,000 Da, at least 6,000 Da, at least 7,000 Da, or at least 8,000 Da. Each possibility represents a separate embodiment. According to some embodiments, the first polymeric linker has a MW of at most 2,000 Da, at most 3,000 Da, at most 4,000 Da, at most 5,000 Da, at most 6,000 Da, at most 7,000 Da, or at most 10,000 Da. Each possibility represents a separate embodiment.

In some embodiments, the second polymeric linker comprises a polymer selected from the group consisting of a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives and combinations thereof. Each possibility represents a separate embodiment of the invention.

In some embodiments, the second polymeric linker comprises a polyether. In some embodiments, the second polymeric linker is a polyether. In some embodiments, the polyether is polyethylene glycol (PEG) or a derivative thereof.

In some embodiments, the second polymeric linker comprises a thiol (—SH) end group. In some embodiments, said second polymeric linker is chemically attached to the core particle through the thiol (—SH) end group. In some embodiments, the second polymeric linker is conjugated to the second active agent through an amide bond. In some embodiments, the core particle is bound to the second polymeric linker through a sulfide bond and the second active agent is conjugated to said second polymeric linker through an amide bond. In some embodiments, the core particle is an inorganic particle and is bound to the second polymeric linker through a sulfide bond and the second active agent is conjugated to said second polymeric linker through an amide bond. In some embodiments, the second polymeric linker within the co-delivery system has a structure —S—R—CONH—, wherein R is a polymeric chain consisting of repeating monomer units. In other embodiments, the second polymeric linker within the co-delivery system has a structure —S—R—NHCO—, wherein R is a polymeric chain consisting of repeating monomer units. In some embodiments, the second polymeric linker is selected from thiolated PEG acid (HS-PEG-COOH) and thiolated PEG amine (HS-PEG-NH₂). It is to be understood that the HS and COOH/NH₂ end groups refer to the polymeric linker prior to conjugation with the core particle and the active agent. In some embodiments, the thiol group is chemically attached to the core particle and the acid or amine group is covalently conjugated to the second active agent. In some embodiments, the second polymeric linker within the co-delivery system has a structure selected from —S-PEG-C(O)-and-S-PEG-NH—.

In some embodiments, the second polymeric linker is a non-cleavable linker. In some embodiments, the second polymeric linker is non-cleavable under physiological conditions.

In some embodiments, the second polymeric linker has a molecular weight (MW) between 2,000 to 7,000 Da. In some embodiments, the second polymeric linker has an MW within a range selected from the group consisting of 500-10,000 Da, 600-9,500 Da, 700-9,000 Da, 800-8,500 Da, 800-6,000 Da, 800-5,000 Da, 800-4,000 Da, 800-3,000 Da, 800-2,000 Da, 900-8,000 Da, 1,000-7,000 Da, 1,500-6,500 Da, 2,000-6,000 Da, 3,000-6,000 Da, 4,000-6,000 Da, 1,000-2,000 Da, 1,000-3,000 Da, 1,000-4000 Da, 1,000-5,000 Da, 1,000-7,000 Da, 1,000-10,000 Da, 2,000-3,000 Da, 2,000-5,000 Da, 2,000-7,000 Da, 2,000-10,000 Da, 3,000-10,000 Da, 3,000-7,000 Da, 3,000-5,000 Da, 3,000-3,400 Da, 3,400-7,000 Da, 5,000-7,000 Da, 5,000-10,000 Da, and 7,000-10,000 Da. Each possibility represents a separate embodiment. According to some embodiments, the second polymeric linker has an MW of at least 1,000 Da, at least 1,500 Da, at least 2,000 Da, at least 2,500 Da, at least 3,000 Da, at least 3,400 Da, at least 4,000 Da, at least 5,000 Da, at least 6,000 Da, at least 7,000 Da or at least 8,000 Da. Each possibility represents a separate embodiment. According to some embodiments, the second polymeric linker has an MW of at most 2,000 Da, at most 3,000 Da, at most 4,000 Da, at most 5,000 Da, at most 6,000, at most 7,000 Da or at most 10,000 Da. Each possibility represents a separate embodiment.

According to some embodiments, the first polymeric linker and the second polymeric linker comprise different polymers. According to some embodiments, the first polymeric linker and the second polymeric linker are different polymers. In some embodiments the first polymeric linker and the second polymeric linker comprise the same polymer. In some embodiments the first polymeric linker and the second polymeric linker are identical.

In some embodiments, the first and second polymeric linkers comprise the same polymer selected from the group consisting of a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives and combinations thereof. In some embodiments, both the first and second polymeric linkers comprise PEG. In some embodiments, both the first and second polymeric linkers are PEG. In some embodiments, both the first and second polymeric linkers comprise thiolated PEG.

In some embodiments, the first and second polymeric linkers comprise thiolated PEG acid (HS-PEG-COOH) or thiolated PEG amine (HS-PEG-NH₂). In some embodiments, the first and second polymeric linkers are thiolated PEG acid (HS-PEG-COOH) or thiolated PEG amine (HS-PEG-NH₂). In some embodiments, the first and second polymeric linkers are both thiolated PEG acid (HS-PEG-COOH). In some embodiments, the first and second polymeric linkers are both thiolated PEG amine (HS-PEG-NH₂).

In some embodiments, the third polymeric linker comprises a polymer selected from the group consisting of a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives and combinations thereof. Each possibility represents a separate embodiment of the invention.

In some embodiments, the third polymeric linker comprises a polyether. In some embodiments, the third polymeric linker is a polyether. In some embodiments, the polyether is polyethylene glycol (PEG) or a derivative thereof.

In some embodiments, the third polymeric linker comprises a thiol (—SH) end group. In some embodiments, said third polymeric linker is chemically attached to the core particle through the thiol (—SH) end group. In some embodiments, the third polymeric linker is conjugated to the brain internalizing transporter moiety through an amide bond. In some embodiments, the core particle is bound to the third polymeric linker through a sulfide bond and the brain internalizing transporter moiety is conjugated to said third polymeric linker through an amide bond. In some embodiments, the core particle an inorganic particle and is bound to the third polymeric linker through a sulfide bond and the brain internalizing transporter moiety is conjugated to said third polymeric linker through an amide bond. In some embodiments, the third polymeric linker within the co-delivery system has a structure —S—R—CONH—, wherein R is a polymeric chain consisting of repeating monomer units. In other embodiments, the third polymeric linker within the co-delivery system has a structure —S—R—NHCO—, wherein R is a polymeric chain consisting of repeating monomer units. In some embodiments, the third polymeric linker is selected from thiolated PEG acid (HS-PEG-COOH) and thiolated PEG amine (HS-PEG-NH₂). It is to be understood that the HS and COOH/NH₂ end groups refer to the polymeric linker prior to conjugation with the core particle and the brain internalizing transporter moiety. In some embodiments, the thiol group is chemically attached to the core particle and the acid or amine group is covalently conjugated to the brain internalizing transporter moiety. In some embodiments, the third polymeric linker within the co-delivery system has a structure selected from —S-PEG-C(O)— and —S-PEG-NH—.

In some embodiments, the third polymeric linker has a molecular weight (MW) between 2,000 to 7,000 Da. In some embodiments, the third polymeric linker has an MW within a range selected from the group consisting of 2,000-10,000 Da, 2,000-9,500 Da, 2,000-9,000 Da, 2,000-8,500 Da, 2,000-6,000 Da, 2,000-5,000 Da, 2,000-4,000 Da, 2,000-3,000 Da, Da, 2,000-8,000 Da, 2,000-7,000 Da, 2,000-6,500 Da, 2,000-6,000 Da, 3,000-6,000 Da, 4,000-6,000 Da, 2,000-3,000 Da, 2,000-4,000 Da, 2,000-5,000 Da, 2,000-7,000 Da, 2,000-11,000 Da, 2,000-3,000 Da, 2,000-5,000 Da, 2,000-7,000 Da, 2,000-10,000 Da, 3,000-10,000 Da, 3,000-7,000 Da, 3,000-5,000 Da, 3,000-3,400 Da, 3,400-7,000 Da, 5,000-7,000 Da, 5,000-10,000 Da, and 7,000-10,000 Da. Each possibility represents a separate embodiment. According to some embodiments, the third polymeric linker has an MW of at least 2,000 at least 2,500 Da, at least 3,000 Da, at least 3,400 Da, at least 4,000 Da, at least 5,000 Da, at least 6,000 Da, at least 7,000 Da or at least 8,000 Da. Each possibility represents a separate embodiment. According to some embodiments, the third polymeric linker has an MW of at most 2,000 Da, at most 3,000 Da, at most 4,000 Da, at most 5,000 Da, at most 6,000, at most 7,000 Da or at most 10,000 Da. Each possibility represents a separate embodiment.

In some embodiments, the third polymeric linker is a non-cleavable linker. In some embodiments, the third polymeric linker is non-cleavable under physiological conditions.

In some embodiments, at least one of the first, the second and the third polymeric linkers, or an additional polymeric linker comprises a cleavable linker. In some embodiments, at least one of the first and the second polymeric linkers comprises a cleavable linker. In some embodiments, each one of the first and the second polymeric linkers independently comprises a cleavable linker. According to some embodiments, the cleavable linker is SH-PEG-SH. According to some embodiments, the cleavable linker comprises a bond susceptible to cleavage by an endogenous molecule, located or expressed in the brain. In some embodiments, the cleavable linker is PEG succinimidyl succinate (PEGSS). According to some embodiments, the endogenous molecule is glutathione. According to some embodiments, the endogenous molecule is selected from the group comprising of proteases, nucleases, hydronium ions, and reducing agents. In some embodiments, the endogenous molecule is selected from neuroserpin and Serpin B. Each possibility represents a separate embodiment. According to some embodiments, the cleavable linker connects a chemotherapeutic molecule or a toxin to the multifunctional particle.

Any chemotherapeutic molecule or toxin which are known in the art to have anticancer activity may be used in the multifunctional delivery systems of the present invention. The chemotherapeutic molecule include but is not limited to: irinotecan, deruxtecan, emtansine, mitoxantrone, topoisomerase inhibitors, spindle poison from vinca: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabine; podophyllotoxins: etoposide, topotecan, dacarbazine; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, and megestrol acetate. Each possibility represents a separate embodiment of the invention.

According to some embodiments, the chemotherapeutic agent is selected from alkylating agents, antimetabolites, folic acid analogs, pyrimidine analogs, purine analogs and related inhibitors, vinca alkaloids, epipodophyllotoxins, antibiotics, L-asparaginase, topoisomerase inhibitor, interferons, platinum coordination complexes, anthracenedione substituted urea, methyl hydrazine derivatives, adrenocortical suppressant, adrenocorticosteroids, progestins, estrogens, antiestrogen, androgens, antiandrogen, and gonadotropin-releasing hormone analog. According to another embodiment, the chemotherapeutic agent is selected from the group consisting of 5-fluorouracil (5-FU), leucovorin (LV), irinotecan, oxaliplatin, capecitabine, paclitaxel and docetaxel. One or more chemotherapeutic agents can be used with the multifunctional delivery system of the present invention. Each possibility represents a separate embodiment of the invention.

According to some embodiments, the toxin is selected from microtubule inhibitor, DNA synthesis inhibitor, topoisomerase inhibitor and RNA polymerase inhibitor. According to additional embodiments, the toxin is selected from the group consisting of MMAE, MMAF, Saporin, DM4, DM1, SN-38, Calicheamicin, DXd, PBD, Duocarmycin, Sandramycin, alpha-Amanitin, Chaetocin, CYT997, Daunorubicin, 17-AAG, Agrochelin A, Doxorubicin, Methotrexate, Colchicine, Cordycepin, Epothilone B, Hygrolidin, Herboxidiene, Ferulenol, Curvulin, paclitaxel, Englerin A, Taltobulin, Triptolide, Cryptophycin, and Nemorubicin. Each possibility represents a separate embodiment of the invention.

According to some embodiments, the multifunctional particle further comprises a cleaving molecule inducer. According to some embodiments, the cleaving molecule inducer is selected from the group comprising of N-acetyl−1-cysteine (NAC), glutathione monoester, γ-glutamylcysteine, γ-glutamylcysteine synthetase, glutathione synthetase. Each possibility represents a separate embodiment.

In some embodiments, the endogenous molecule is glutathione and the cleaving molecule inducer is selected from the group comprising of N-acetyl−1-cysteine (NAC), glutathione monoester, γ-glutamylcysteine, γ-glutamylcysteine synthetase, glutathione synthetase.

According to some embodiments, at least one of the first polymeric linker and the second polymeric linker is different than the third polymeric linker. In some embodiments, at least one of the first polymeric linker and the second polymeric linker comprises the same polymer as the third polymeric linker. In some embodiments, the first polymeric linker, the second polymeric linker and the third polymeric linker comprise the same polymer. In further embodiments, the first polymeric linker is composed of repeating monomer units and the third polymeric linker is composed of the same repeating monomer units as the first linear polymeric linker. In some related embodiments, the first linear polymeric linker has a different number of repeating monomer units than the third linear polymeric linker. In some embodiments, the second polymeric linker is composed of repeating monomer units and the third polymeric linker is composed of the same repeating monomer units as the second linear polymeric linker. In some related embodiments, the second linear polymeric linker has a different number of repeating monomer units than the third linear polymeric linker. In some embodiments, the first and the second polymeric linkers are identical and are composed of repeating monomer units, and the third polymeric linker is composed of the same repeating monomer units as the first and the second linear polymeric linkers. In some related embodiments, the first and the second linear polymeric linkers have a different number of repeating monomer units than the third linear polymeric linker.

In some embodiments, the first, the second and the third polymeric linkers comprise the same polymer selected from the group consisting of a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives and combinations thereof. In some embodiments, the first, the second and the third polymeric linkers comprise PEG. In some embodiments, the first, the second and the third polymeric linkers are PEG. In some embodiments, the first, the second and the third polymeric linkers comprise thiolated PEG. In some embodiments, the first, the second and the third polymeric linkers comprise thiolated PEG acid (HS-PEG-COOH) or thiolated PEG amine (HS-PEG-NH₂). In some embodiments, the first, the second and the third polymeric linkers are thiolated PEG acid (HS-PEG-COOH) or thiolated PEG amine (HS-PEG-NH₂). In some embodiments, the first, the second and the third polymeric linkers are thiolated PEG acid (HS-PEG-COOH). In some embodiments, the first, the second and the third polymeric linkers are thiolated PEG amine (HS-PEG-NH₂).

In some embodiments, the first active agent is covalently conjugated to the first polymeric linker through a first functional end group of said linker, the second active agent is covalently conjugated to the second polymeric linker through a second functional end group of said linker, and the brain-internalizing transporter moiety is covalently conjugated to the third polymeric linker through a third functional end group of said linker. Exemplary functional end group include but not limited to thiol group, carboxylic group, and amine group. In some embodiments, at least two of the first functional end group, the second functional end group and the third functional end group, are identical. In some embodiments, the first functional end group and the second functional end group are identical. In some embodiments, the first functional end group and the third functional end group are identical. In some embodiments, the second functional end group and the third functional end group are identical. In some embodiments, the first functional end group, the second functional end group and the third functional end group are identical.

In some embodiments, the first functional end group and the second functional end group are different. In some embodiments, the first functional end group and the third functional end group are different. In some embodiments, the second functional end group and the third functional end group are different.

In some embodiments, the first, the second and the third polymeric linkers are linear. According to the principles of the present invention, the length of the third polymeric linker is substantially different than the length of at least one of the first and the second polymeric linkers. In some embodiments, the length of the third polymeric linker is substantially different than the length of the first polymeric linker. In some embodiments, the length of the third polymeric linker is substantially different than the length of the second polymeric linker. In some embodiments, the length of the third polymeric linker is substantially different than the length of both the first polymeric linker and the second polymeric linker. In some embodiments, the length of the first polymeric linker is substantially similar to the length of the second polymeric linker and the length of the third polymeric linker is substantially different than the length of both the first polymeric linker and the second polymeric linker.

In some embodiments, the term “length” of a polymeric moiety or linker refers to the length of the polymer which depends on the number of monomers incorporated therein, the length of each monomer unit, the polymer chain structure (for example, whether the polymer is linear or branched), spatial conformation, deformation of valent (or binding angels) angles, and the degree of stretching or coiling.

The length of a polymer can be calculated as known in the art, for example as described in Introduction to Physical Polymer Science, Fourth Edition, L. H. Sperling, First published:4 Nov. 2005, Chapter 3. Additionally, various computational modeling methods, which can be performed using, inter alia, Hyperchem, ACD/3D, MOE 2010.10, or Chem 3D software. can be used for evaluating the length of a polymer, as known in the art. Physical characterization methods, such as, for example, light scattering, can also be used to assess the length of a polymer. It is to be understood that when assessing the difference between the length of polymeric linkers, the same length definitions (or length measurement methods) must be used for the compared polymeric linkers.

The term “length” when referring to a linear polymer can refer to different length definitions. According to some embodiments, the term “length” refers to a displacement length, also termed herein “end-to-end” length, which is the distance between two ends of the polymer chain for a coiled polymer. End-to-end length can be expressed, for example, as Flory radius:

$$F = α n \frac{3}{5} \qquad Equation I$$

wherein F=Flory radius, a=monomer dimension, n=degree of polymerization,

According to some embodiments, the term “length” refers to contour length, which is the distance between two ends of the polymer chain when the polymer is stretched out. The contour length could be considered the maximum possible displacement length. Contour length (also termed herein “old contour length”) can be calculated by dividing MW of the polymer by the MW of the monomer unit and multiplying by the length of the monomer unit. To account for binding angles, the contour length (also termed herein “new contour length”) can be calculated by dividing MW of the polymer by the MW of the monomer unit, multiplying by the length of the monomer unit and further multiplying by cosine of the ((binding angle theta-180)/2).

As explained hereinabove, the length of a linear polymer can be estimated based on its molecular weight and chemical structure of a monomer unit. In order to evaluate the difference between the polymeric linkers which comprise the same polymer (i.e., composed of the same type but a different number of monomer units), molecular weights of the polymeric linkers can conveniently be used. Accordingly, in some embodiments, the molecular weight of the third polymeric linker is substantially different than the molecular weights of at least one of the first and the second linear polymeric linkers. In some embodiments, the molecular weight of the third polymeric linker is substantially different than the molecular weight of the first polymeric linker. In some embodiments, the molecular weight of the third polymeric linker is substantially different than the molecular weight of the second polymeric linker. In some embodiments, the molecular weight of the third polymeric linker is substantially different than the molecular weights of the first and the second polymeric linker.

As used herein, the term “substantially different” refers to a difference of at least 5%, at least 6%, at least 7%, at least 8%, at least 9%, at least 10%, at least 12%, at least 15%, at least 18%, at least 20%, at least 25%, at least 30%, at least 35%, at least 40%, at least 45%, or at least 50%. Each possibility represents a separate embodiment of the present invention. The term “substantially higher” means that a first value is higher than a second value wherein the difference between the first and the second values is at least 5%, at least 6%, at least 7%, at least 8%, at least 9%, at least 10%, at least 12%, at least 15%, at least 18%, at least 20%, at least 25%, at least 30%, at least 35%, at least 40%, at least 45%, or at least 50%. Each possibility represents a separate embodiment of the present invention.

In some embodiments, the molecular weight of the monomeric unit of the third polymeric linker is substantially similar to the molecular weight of the monomeric unit of the at least one of the first and the second polymeric linkers. As used herein, the term “substantially similar” refers to a similarity of at least 70%, at least 75%, at least 80%, at least 85%, at least 90%, at least 91%, at least 92%, at least 93%, at least 94%, or at least 95%. Each possibility represents a separate embodiment of the present invention.

In some embodiments, the third polymeric linker and at least one of the first polymeric linker and the second polymeric linker comprise the similar polymer. In some embodiments, the third linear polymeric linker is composed of repeating monomer units and at least one of the first polymeric linker and the second linear polymeric linker is composed of the same repeating monomer units as the third linear polymeric linker, wherein the third linear polymeric linker has a different number of repeating monomer units than the at least one of the first polymeric linker and the second linear polymeric linker. In some embodiments, the third polymeric linker and the at least one of the first polymeric linker and the second polymeric linker are similar except for the length of said third and said first and/or second polymeric linkers.

In some embodiments, the third polymeric linker and the at least one of the first polymeric linker and the second polymeric linker, have a difference in their respective molecular weights of at least about 100 Da, at least about 150 Da, at least about 200 Da, at least about 250 Da, at least about 300 Da, at least about 350 Da, at least about 400 Da, at least about 450 Da, at least about 500 Da, at least about 550 Da, at least about 600 Da, at least about 650 Da, at least about 700 Da, at least about 750 Da, at least about 800 Da, at least about 850 Da, at least about 900 Da, at least about 950 Da, at least about 1000 Da, at least about 1100 Da, at least about 1200 Da, at least about 1300 Da, at least about 1400 Da, at least about 1500 Da, at least about 1600 Da, at least about 1700 Da, at least about 1800 Da, at least about 1900 Da, or at least about 2000 Da. Each possibility represents a separate embodiment of the present invention.

In some embodiments, the difference between the lengths of third polymeric linker and the at least one of the first polymeric linker and the second linear polymeric linker, is configured to enable exposure of the brain internalizing transporter moiety on the external surface of the co-delivery system, which faces the BBB. It is to be understood that the active agents are not enclosed or encapsulated within the core particle, but rather are attached to the external surface thereof, via a polymeric linker, similarly to the brain-internalizing moiety, which is also attached to the surface of the same core particle via a polymeric linker. Without wishing to being bound by theory or mechanism of action, it is contemplated that attaching the brain internalizing transporter moiety through a polymeric chain having similar length as the first and/or second polymeric linkers, may prevent sufficient exposure of said brain-internalizing moiety on the external surface of the co-delivery system, and thereby limit the penetration of the system through the BBB.

Without further wishing to being bound by theory or mechanism of action, it is contemplated that the active agents, which are not enclosed or encapsulated within the core particle, remain accessible and active despite being bound to the multifunctional system. Advantageously, the specific composition of the multifunctional system of the invention which ensures formation of a conjugated particle with a particular hierarchical structure, not only allows to deliver various types of active agents combinations, but also does not interfere with the functionality of the active agents, such that cleavage of the linkage between at least one of the active agents and the core particle after penetration through the BBB, is not necessarily required.

Accordingly, in some embodiments, the molecular weight of the third polymeric linker is higher than the molecular weight of at least one of the first and the second polymeric linker. In further embodiments, the molecular weight of the third polymeric linker is higher than the molecular weight of both the first and the second polymeric linkers. In some embodiments, the molecular weight of the third polymeric linker is higher than the molecular weight of the first and/or the second polymeric linker provided that the molecular weight of said first and/or the second polymeric linker is less than 4950 Da. In some embodiments, the molecular weight of the third polymeric linker is higher than the molecular weight of the first and/or the second polymeric linker provided that the molecular weight of said first and/or the second polymeric linker is less than 4900 Da. In some embodiments, the molecular weight of the third polymeric linker is higher than the molecular weight of the first and/or the second polymeric linker provided that the molecular weight of said first and/or the second polymeric linker is less than 4800 Da. In some embodiments, the molecular weight of the third polymeric linker is higher than the molecular weight of the first and/or the second polymeric linker provided that the molecular weight of said first and/or the second polymeric linker is less than 4780 Da. In some embodiments, the third polymeric linker is a PEG derivative having a molecular weight of about 5000 Da and at least one of the first and the second polymeric linkers is a PEG derivative having a molecular weight of about 3500 kDa. In some embodiments, the third polymeric linker is a PEG derivative having a molecular weight of about 5000 Da, and the first and the second polymeric linkers are both PEG derivatives having a molecular weight of about 3500 kDa.

In some embodiment, the third polymeric linker has a molecular weight which is higher than the molecular weight of at least one of the first and the second polymeric linkers. In some embodiments, the MW of the polymeric linkers directly depend on the relative molecular weights of the active molecules and the brain internalizing moiety. In some embodiments, the first active molecule has a higher MW than the brain internalizing moiety and the first polymeric linker has a lower MW than the third polymeric linker. In some embodiments, the second active molecule has a higher MW than the brain internalizing moiety and the second polymeric linker has a lower MW than the third polymeric linker.

In some embodiment, the third polymeric linker is longer than the first and/or the second polymeric linker. In some embodiments, the third polymeric linker has a higher end-to-end distance than the first and/or the second polymeric linker. In some embodiments, the third polymeric linker has a higher contour distance than the first and/or the second polymeric linker.

In some embodiments, the third polymeric linker has a MW lower than the MW of at least one of the first and the second polymeric linkers. In some related embodiments, the MW of the at least one of the first and the second polymeric linkers is at least about 4000 Da. In further related embodiments, the difference between the Mw of the first polymeric linker and the at least one of the first and the second polymeric linkers is at least about 2000 Da. Without wishing to being bound by theory or mechanism of action, it is contemplated that the significantly longer first and/or second linker allows folding of the polymer chain (or a higher degree of coiling), such that the actual distance between the respective active agent and the core particle is smaller than between the brain-internalizing moiety and the core particle, such that the active agent is at least partly shielded by the brain-internalizing moiety which is exposed on the surface of the multifunctional particle during BBB penetration. In some related embodiments, the end-to-end distance of the third polymeric linker is higher than the end-to-end distance of the first and/or the second polymeric linker, despite the higher MW of said first and/or second polymeric linker.

In some embodiments, the distance between the first active agent and the core particle and the distance between the second active agent and the core particle, are smaller than the distance between the brain-internalizing moiety and the core particle. In some embodiments, at least one end group of the third polymeric linker is similar to at least one end group of the first polymeric linker. In some embodiments, at least one end group of the third polymeric linker is similar to at least one end group of the second polymeric linker. In some embodiments, the two end groups of the third polymeric linker are similar to the two end groups of the first polymeric linker. In some embodiments, the two end groups of the third polymeric linker are similar to the two end groups of the second polymeric linker. In some embodiments, the two end groups of the first polymeric linker are similar to the two end groups of the second polymeric linker.

In some embodiments, the core particle is bound to an additional, fourth, polymer. In some embodiments, said polymer is a monofunctional polymeric linker. In some embodiments, the core particle is coated with a polymeric layer comprising the first polymeric linker, the second polymeric linker, the third polymeric linker and additional, fourth, polymeric linker wherein the additional polymeric linker is monofunctional and used for capping functional groups on the particles and to enable sufficient distance between the other linkers and active molecules and the transporter. The terms “fourth polymer” and “fourth polymeric linker” can be used interchangeably. In some embodiments, the fourth polymer functions as a spacer moiety. In some embodiments, the fourth polymeric linker is a linear polymeric linker. In some embodiments, the fourth polymer is selected from the group consisting of a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives and combinations thereof.

As used herein, the term “monofunctional” means that the polymer before being conjugated to the core particle has only one functional group configured to bind said polymer to the core particle. The monofunctional polymeric linker is therefore neither conjugated nor capable of conjugating any moiety except for the core particle and it is used as a capping moiety.

In some embodiments, the fourth polymer comprises the same monomer units as the first and/or the second polymers. In some embodiments, the fourth polymer comprises the same monomer units as the third polymeric linker. In some embodiments, the first, the second, the third and the fourth polymers comprise the same monomer units. In some embodiments, the fourth polymer is bound to the core particle through a thiol end group of said polymer. In some embodiments, the fourth polymer is a polyether. In some embodiments, the polyether is methoxy polyethylene glycol (mPEG) or a derivative thereof. In some embodiments, the mPEG is thiolated (mPEG-SH) wherein said thiolated mPEG is bound to the core particle via the thiol end group.

In some embodiments, the fourth polymer has a MW between 1,000 to 7,000 Da. In some embodiments, the fourth polymer has a MW from 500-1,000 Da, 500-3,000 Da, 500-7,000 Da, 500-10,000 Da, 1,000-3,000 Da, 1,000-4,000 Da, 1,000-5,000 Da, 1,000-7,000 Da, 1,000-10,000 Da, 3,000-5,000 Da, 3,000-7,000 Da, 3,000-10,000 Da, 7,000-10,000 Da. Each possibility represents a separate embodiment. According to some embodiments, the fourth polymer has a MW of at least 1,000 Da, at least 2,000 Da, at least 3,000 Da, at least 4,000 Da, at least 5,000 Da, at least 6,000 Da, at least 7,000 Da, or at least 8,000 Da. Each possibility represents a separate embodiment. According to some embodiments, the fourth polymer has a MW of at most 1,000 Da, at most 2,000 Da, at most 3,000 Da, at most 4,000 Da, at most 5,000 Da, at most 6,000 Da, at most 7,000 Da, or at most 10,000 Da. Each possibility represents a separate embodiment.

In some embodiments, the length of the fourth polymer is substantially similar to the length of at least one of the first polymeric linker, the second polymeric linker and the third polymeric linker. In some embodiments, the length of the fourth polymer is substantially similar to the length of the first polymeric linker. In some embodiments, the length of the fourth polymer is substantially similar to the length of the second polymeric linker. In some embodiments, the length of the fourth polymer is substantially similar to the length of the third polymeric linker. In some embodiments, the length of the fourth polymer is substantially similar to the length of the polymeric linker (first, second or third) that its length is higher than the length of at least one of the other polymeric linkers. In some embodiments, the molecular weight of the fourth polymer is substantially similar to the molecular weight of the polymeric linker (first, second or third) having higher molecular weight than at least one of the other polymeric linkers. In some embodiments, the MW of the fourth polymer is substantially similar to the MW of the first polymeric linker. In some embodiments, the MW of the fourth polymer is substantially similar to the MW of the second polymeric linker. In some embodiments, the MW of the fourth polymer is substantially similar to the MW of the third polymeric linker.

Without wishing to being bound by theory or mechanism of action, the efficacy of the co-delivery system of the invention also depends on molar ratio of the different polymeric linkers, wherein said ratio defines the density of the brain internalizing transporter moiety and the active agents within the co-delivery system.

In some embodiments, the first polymeric linker constitutes about 5-70% mol, 5-60% mol, 5-40% mol, 8-60% mol, 10-60% mol, 10-55% mol, 10-50% mol, 10-40% mol, 10-30% mol, 10-25% mol, 10-20% mol, 15-60% mol, 15-55% mol, 15-50% mol, 15-45% mol, 15-40% mol, 15-30% mol, 15-25% mol, 15-20% mol, 2-10% mol, 2-20% mol, 2-50% mol, 2-60% mol, 2-70% mol, 5-10% mol, 5-20% mol, 5-70% mol, 10-20% mol, 10-50% mol, 10-70% mol, 20-50% mol, 20-40% mol, 30-50% mol, 30-60% mol, 30-70% mol, 50-60% mol or 50-70% mol of the total polymers bound to the core particle. Each possibility represents a separate embodiment of the present invention. In some embodiments, first polymeric linker constitutes at least 2% mol, at least 4% mol, at least 5% mol, at least 6% mol, at least 8% mol, at least 10% mol, at least 12% mol, at least 15% mol, at least 18% mol, at least 20% mol, at least 25% mol, at least 30% mol, at least 35% mol, at least 40% mol, at least 50% mol, or at least 60% mol of the total polymers bound to the core particle. Each possibility represents a separate embodiment.

In some embodiments, the second polymeric linker constitutes about 5-70% mol, 5-60% mol, 5-40% mol, 8-60% mol, 10-60% mol, 10-55% mol, 10-50% mol, 10-40% mol, 10-30% mol, 10-25% mol, 10-20% mol, 15-60% mol, 15-55% mol, 15-50% mol, 15-45% mol, 15-40% mol, 15-30% mol, 15-25% mol, 15-20% mol, 2-10% mol, 2-20% mol, 2-50% mol, 2-60% mol, 2-70% mol, 5-10% mol, 5-20% mol, 5-70% mol, 10-20% mol, 10-50% mol, 10-70% mol, 20-50% mol, 20-40% mol, 30-50% mol, 30-60% mol, 30-70% mol, 50-60% mol or 50-70% mol of the total polymers bound to the core particle. Each possibility represents a separate embodiment of the present invention. In some embodiments, the second polymeric linker constitutes at least 2% mol, at least 4% mol, at least 5% mol, at least 6% mol, at least 8% mol, at least 10% mol, at least 12% mol, at least 15% mol, at least 18% mol, at least 20% mol, at least 25% mol, at least 30% mol, at least 35% mol, at least 40% mol, at least 50% mol, or at least 60% mol of the total polymers bound to the core particle. Each possibility represents a separate embodiment.

In some embodiments, the third polymeric linker constitutes about 5-70% mol, 5-60% mol, 5-40% mol, 8-60% mol, 10-60% mol, 10-55% mol, 10-50% mol, 10-40% mol, 10-30% mol, 10-25% mol, 10-20% mol, 15-60% mol, 15-55% mol, 15-50% mol, 15-45% mol, 15-40% mol, 15-30% mol, 15-25% mol, 15-20% mol, 2-10% mol, 2-20% mol, 2-50% mol, 2-60% mol, 2-70% mol, 5-10% mol, 5-20% mol, 5-70% mol, 10-20% mol, 10-50% mol, 10-70% mol, 20-50% mol, 20-40% mol, 30-50% mol, 30-60% mol, 30-70% mol, 50-60% mol or 50-70% mol of the total polymers bound to the core particle. Each possibility represents a separate embodiment of the present invention. In some embodiments, the third polymeric linker constitutes at least 2% mol, at least 4% mol, at least 5% mol, at least 6% mol, at least 8% mol, at least 10% mol, at least 12% mol, at least 15% mol, at least 18% mol, at least 20% mol, at least 25% mol, at least 30% mol, at least 35% mol, at least 40% mol, at least 50% mol, or at least 60% mol of the total polymers bound to the core particle. Each possibility represents a separate embodiment.

In some embodiments, the fourth polymer constitutes about 5-90% mol, 5-85% mol, 5-80% mol, 10-80% mol, 20-78% mol, 25-75% mol, 30-75% mol, 40-75% mol, 50-75% mol, 60-75% mol, 60-70% mol, 60-80% mol, 5-60% mol, 10-60% mol, 10-55% mol, 10-50% mol, 10-40% mol, 15-60% mol, 15-55% mol, 15-50% mol, 15-45% mol or 15-40% mol of the total polymers bound to the core particle. Each possibility represents a separate embodiment of the present invention.

In some embodiments, the fourth polymer constitutes between 60-80% mol of the total polymers bound to the core particle. In some embodiments, the fourth polymer constitutes between 50-80% mol of the total polymers bound to the core particle. In some embodiments, the fourth polymer constitutes at least 2% mol, at least 4% mol, at least 5% mol, at least 6% mol, at least 8% mol, at least 10% mol, at least 12% mol, at least 15% mol, at least 18% mol, at least 20% mol, at least 25% mol, at least 30% mol, at least 35% mol, at least 40% mol, at least 45% mol, at least 50% mol, at least 55% mol, at least 60% mol, at least 65% mol, or at least 70% mol of the total polymers bound to the core particle. Each possibility represents a separate embodiment.

In some embodiments, the first polymeric linker constitutes about 5 to 45% mol, the second polymeric linker constitutes about 5 to 45% mol, the third polymeric linker constitutes about 10 to 45% mol, and the fourth polymer constitutes about 40 to 80% mol of the total polymers bound to the core particle.

In some embodiments, the first polymeric linker constitutes about 10 to 40% mol, the second polymeric linker constitutes about 10 to 40% mol, the third polymeric linker constitutes about 10 to 40% mol, and the fourth polymer constitutes about 40 to 70% mol of the total polymers bound to the core particle.

In some embodiments, the first and second polymeric linkers together constitute about 10% to 60% mol, 10 to 50% mol, 10 to 45% mol, 10 to 40% mol, 10 to 30% mol or 10 to 20% mol of the total polymeric linkers bound to the core particle. Each possibility represents a separate embodiment of the present invention.

It is to be understood that the % mol of each polymer is dependent on the other polymers bound to the core particle, such that the total % mol of the polymers does not exceed 100%.

In some embodiments, the first polymeric linker, second polymeric linker, third polymeric linker and fourth polymer are in a (w/w/w/w) ratio of between 5:5:5:85 to 20:20:30:30.

According to the principles of the present invention, the co-delivery system comprises a brain-internalizing transporter moiety conjugated to the third polymeric linker. The term “brain-internalizing transporter moiety”, which can be used interchangeably herein with the term “brain-internalizing moiety” refers to a molecule that can specifically bind to a receptor or surface protein expressed by a cellular component of the BBB. The three major cellular elements of the brain microvasculature, which collectively form the BBB, are brain endothelial cells, astrocyte end feet and pericytes (PCs). In some embodiments, the brain-internalizing transporter moiety can bind to a receptor or surface protein expressed by a brain endothelial cell. In some embodiments, the brain-internalizing transporter moiety can bind to a receptor or surface protein expressed by astrocyte end feet. In some embodiments, the brain-internalizing transporter moiety can bind to a receptor or surface protein expressed by pericytes (PCs). Without wishing to be bound by any theory or mechanism, it is hypothesized that the brain internalizing moiety promotes the transportation of the entire co-delivery system through the BBB, possibly through a receptor mediated transcytosis (RMT) or receptor mediated endocytosis (RME) mechanism.

In some embodiments, the brain-internalizing moiety is selected from, but not limited to, insulin, an antibody specific for the insulin receptor, or part of such antibody-such as Fab fragment, transferrin, an antibody specific for the transferrin receptor, or part of such antibody, a polypeptide that specifically binds to the transferrin receptor, a polypeptide that specifically binds to the insulin receptor, insulin-like growth factor 1, an antibody specific for the insulin-like growth factor receptor 1, or part of such antibody, a polypeptide that specifically binds to the insulin-like growth factor receptor 1, apolipoprotein Al, B, or E, lactoferrin, angiopep-2, an antibody specific for low density lipoprotein receptor or lipoprotein receptor-related protein, a polypeptide that specifically binds to low density lipoprotein receptor or lipoprotein receptor-related protein, an antibody specific for diphtheria toxin receptor, or part of such antibody, a polypeptide that specifically binds to diphtheria toxin receptor, and a BBB-penetrant cell-penetrating peptide (CPP). Each possibility represents a separate embodiment of the present invention. As used herein, the term “cell-penetrating peptide (CPP)” refers to a peptide that has an enhanced ability to cross cell membrane bilayer without causing a significant lethal membrane damage. The term “BBB-penetrant CPP” refers to a cell-penetrating peptide that can cross the membrane of BBB cells and is therefore able to penetrate into the brain (Zou, Li-li, et al. Current neuropharmacology 11.2 (2013): 197-208, and Stalmans, Sofie, et al. PloS one 10.10 (2015): e0139652).

Other cellular proteins capable of facilitating transcytosis that are known in the art can also be used as a brain-internalizing moiety. In some embodiments, the brain-internalizing moiety is selected from the group consisting of insulin, transferrin, a low-density lipoprotein, apolipoprotein Al, B, or E, and lactoferrin. Each possibility represents a separate embodiment of the present invention. In some embodiments, the brain-internalizing moiety is selected from the group consisting of insulin and transferrin. In some embodiments, the brain-internalizing moiety is insulin. In some embodiments, the molecular weight (MW) of the brain-internalizing moiety is about 5 kilodaltons (kD).

According to the principles of the present invention, the first polymeric linker is conjugated to a first active agent and the second polymeric linker is conjugated to a second active agent. As used herein, the term “active agent” refers to an agent that is intended to be delivered into the brain of a subject and is capable of being used as a therapeutic, targeting or diagnostic agent. In some embodiments, each one of the first active agent and the second active agent is independently selected from a biologically active molecule and a labeling molecule. According to some embodiments, the first active agent is characterized by a poor BBB penetration. According to some embodiments, the second active agent is characterized by a poor BBB penetration. According to some embodiments, the first active agent and the second active agent are characterized by a poor BBB penetration. According to some embodiments, upon penetration through the BBB, the first and/or the second active agent can further target the co-delivery system to a specific region within the brain, e.g., the hippocampus, the striatum, the medulla, the cerebellum and the cortex. According to some embodiments, upon penetration through the BBB, the first and/or the second active agent can target the nano-delivery system to specific cell population inside the brain, e.g., glioma (or other tumor) cells, microglial cells, astrocytes and neuronal cells.

In some embodiments, each one of the first active agent and the second active agent is independently selected from the group consisting of, but not limited to a small molecule, a macromolecule, an oligonucleotide, an antisense RNA, a peptide, a chemical reagent, a toxin and any combination thereof. In some embodiments, each one of the first active agent and the second active agent is independently selected from the group consisting of a macromolecule, a peptide, a toxin and a small molecule. In some embodiments, each one of the first active agent and the second active agent is independently selected from the group consisting of a polypeptide, an antibody, a peptide and a small molecule. Each possibility represents a separate embodiment of the present invention.

An oligonucleotide molecule according to the present invention may include any DNA or RNA molecule, which may be natural, synthetic or modified. The oligonucleotides may be single stranded or double stranded. The oligonucleotides of the present invention may include, but are not limited to, short interfering RNA (siRNA), micro RNA (miRNA), double stranded RNA (dsRNA), antisense RNA or DNA, aptamer oligonucleotides, peptide nucleic acids (PNAs), sugar ring modified oligonucleotides, Nucleoside organothiophosphate (PS) analogs, CpG oligonucleotides, and DNAenzymes.

In some embodiments, the first and the second active agents are of the same type selected from a small molecule, an antibody, an oligonucleotide, an antisense RNA, and a peptide. In some related embodiments, the first polymeric linker and the second polymeric linker are identical.

In some embodiments, the first and/or the second active agent is a biologically active molecule. In some embodiments, the biologically active molecule is contiguous to the respective polymeric linker. The term “biologically active molecule” as used herein, refers to compounds or molecules that are capable of eliciting or modifying a biological response in a system or capable of binding to specific cellular receptors/markers and thereby targeting the system to specific cells. In some embodiments, the biologically active molecule is a therapeutic agent. In some embodiments, the biologically active molecule has therapeutic applications. In some embodiments, the biologically active molecule has diagnostic applications. In some embodiments, the biologically active molecule has both therapeutic and diagnostic applications. In some embodiments, the biologically active molecule comprises a small molecule, a macromolecule, an oligonucleotide, an antisense RNA, a peptide, chemical reagent, or any combination thereof. Each possibility represents a separate embodiment of the present invention.

In some embodiments, the first active agent and/or the second active agent is a macromolecule. The term ‘macromolecule,’ as defined herein refers to a very large molecule, commonly formed via polymerization of monomers. In some embodiments, the macromolecule is a polypeptide or a protein. In some embodiments, the macromolecule is an enzyme. In some embodiments, the macromolecule is an antibody or a fragment thereof. In some specific embodiments, the antibody is selected from the group the group consisting of anti-IgG1, anti-IbA1, anti-HER2+(Trastuzumab & Pertuzumab), anti-EGFR (Cetuximab), anti-GD2 and checkpoint inhibitor antibodies such as anti PD-1, anti PD-L1 and anti-CTLA-4, or a fragment thereof.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜ Fc fusions and scFv-scFv-Fc fusions.

In some embodiments, the first active agent and/or the second active agent is an antibody. In some embodiments, the antibody is an antibody that binds specifically to a receptor present on the surface of target cells in the brain. In some embodiments, the antibody is an antibody that binds specifically to a receptor present on cells in specific brain region. In some embodiments, the antibody is an antibody that binds specifically to a receptor present on the surface of diseased cells in the brain. In some embodiments, the antibody is a bi-specific antibody. In some embodiments, the first and the second active agents are both bi-specific antibodies. In some embodiments, the first active agent and/or the second active agent is an antibody having a therapeutic activity against a brain-related disease or disorder.

Exemplary antibodies include but are not limited to: anti-HER2+(Trastuzumab & Pertuzumab), anti-EGFR (Cetuximab), checkpoint inhibitor antibodies (Anti PD-1, Anti PD-L1, Anti-CTLA-4), and anti-GD2.

In some embodiments, the antibody has a molecular weight (MW) of 100-120 kD, 100-150 kD, 100-200 kD, 100-250 kD, 150-200 kD, 150-250 kD, 200-250 kD. Each possibility represents a separate embodiment. In some embodiments, the antibody has an MW of at least 100 kD, at least 110 kD, at least 120 kD, at least 130 kD, at least 140 kD, at least 150 kD, at least 160 kD, at least 180 kD, at least 200 kD, at least 250 kD. Each possibility represents a separate embodiment. In some embodiments, the antibody has an MW of 150-200 kD. In some embodiments, the antibody has an MW of 130-180 kD. In some embodiments, the antibody has an MW of 140-160 kD.

In some specific embodiments, the antibody has a MW of 150-200 kD and the respective polymeric linker comprises PEG having a MW of at least 1,000 Da, at least 2,000 Da, at least 2,500 Da or at least 3,000 Da. In some embodiments, the antibody has a MW of 150-200 kD and the respective polymeric comprises PEG having a MW of at most 2,000 Da, at most 2,500 Da, at most 3,000 Da, at most 3,500 Da, at most 4,000 Da, at most 5,000 Da or at most 6,000 Da. In some embodiments, the antibody has a MW of 150-200 kD and the respective polymeric linker comprises PEG having a MW of between 1,000 Da to 4,000 Da. In some such embodiments, the brain internalizing moiety is insulin with a MW of 5-6 kD and the third polymeric linker comprises PEG having a MW of at least 4,000 Da.

In some embodiments, the first active agent and/or the second active agent is a peptide. In some embodiments, the peptide can bind specifically to a receptor present on the surface of target cells in the brain. In some embodiments, the peptide can bind specifically to a receptor present on cells in specific brain region. In some embodiments, the peptide can bind specifically to a receptor present on the surface of diseased cells in the brain. In some embodiments, the peptide has a therapeutic activity against a brain-related disease or disorder.

The term “peptide” as used herein refers to any polymer compound produced by amide bond formation between an α-carboxyl group of one D- or L-amino acid and an «-amino group of another D- or L-amino acid.

In some embodiments, the first active agent and/or the second active agent is a small molecule. In some embodiments, the small molecule can bind specifically to a receptor present on the surface of target cells in the brain. In some embodiments, the small molecule can bind specifically to a receptor present on cells in specific brain region. In some embodiments, the small molecule can bind specifically to a receptor present on the surface of diseased cells in the brain. In some embodiments, the small molecule has a therapeutic activity against a brain-related disease or disorder.

The term “small molecule”, as used herein, refers to organic or inorganic molecules either synthesized or found in nature, generally having a molecular weight less than 1000 Da. Also encompassed by the term “small molecule” is any fragment of a peptide, protein, or polypeptide, including native sequences and variants falling within the molecular weight range stated above.

In some embodiments, the first and/or the second active agent is a therapeutic agent that is effective in treating a brain-related disease or disorder. In some embodiments, the first and/or the second active agent is an antibody used for the treatment or diagnosis of a brain-related disease. In some embodiments, the first and/or the second active agent is a small molecule used for the treatment or diagnosis of a brain-related disease. In some embodiments, the small molecule is selected from cisplatin, Lapatinib, Neratinib, and Tucatinib. Each possibility represents a separate embodiment of the present invention.

In some embodiments, at least one of the first and the second active agents is a labeling molecule. The term “labeling molecule”, as used herein, refers to a molecule that is capable of producing a signal detectable by suitable detection means, such as but not limited to radioactive molecules and fluorescent molecules. In some embodiments, the labeling molecule has diagnostic applications. In some embodiments, the labeling molecule is a diagnostic agent. In some embodiments, the labeling molecule comprises a small molecule, a macromolecule, an oligonucleotide, an antisense RNA, a peptide or any combination thereof. In some embodiments, the labeling molecule is a small molecule. In some embodiments, the labeling molecule is an antibody.

In some embodiments, the first and/or the second active agent is a small molecule having a MW lower than 1,000 Daltons (Da). In some embodiments, the small molecule has a MW of 10-50 Da, 10-100 Da, 10-500 Da, 10-1,000 Da, 50-100 Da, 50-500 Da, 50-1,000 Da, 100-300 Da, 100-500 Da, 100-800 Da, 100-1,000 Da, 500-800 Da, 500-1,000 Da, 800-1,000 Da. Each possibility represents a separate embodiment. In some embodiments, the small molecule has a MW less than 1,000 Da, less than 900 Da, less than 800 Da, less than 700 Da, less than 600 Da, less than 500 Da, less than 400 Da, less than 300 Da, less than 200 Da, less than 100 Da. Each possibility represents a separate embodiment. In some embodiments, the small molecule has a MW more than 100 Da, more than 200 Da, more than 300 Da, more than 400 Da, more than 500 Da, more than 600 Da, more than 700 Da, more than 800 Da, more than 900 Da. Each possibility represents a separate embodiment. In some specific embodiments, the first and/or the second active agent is a small molecule and the respective polymeric linker comprises PEG having a MW of at most 3,000 Da, at most 2,500, at most 2,000 Da, at most 1,500 or at most at most 1,000 Da. Each possibility represents a separate embodiment.

In some embodiments, the first and/or the second active agent is an antisense RNA. In some embodiments, the first and/or the second active agent is a drug.

According to the principles of the present invention, the multifunctional system enables the synchronized co-delivery of two active agents into the brain. In some embodiments, at least one of the first and the second active agents has a poor BBB penetration in its original, free, form. In some embodiments, both the first and the second active agents have a poor BBB penetration in their original, free, form.

In some embodiments, each one of the first and the second active agents is a therapeutic agent having a therapeutic activity against a brain related disease or disorder. One of the advantages of co-delivery systems is the possibility to induce synergistic effects. Regarding co-delivery of different active agents, the therapeutic results can either be additive (i.e., the result is that expected by combining the effects of each drug separately) or synergistic (i.e., the combination produces more-significant benefits than that expected by adding the separate effects). In some embodiments, the combination of the first and the second active agents produces an additive therapeutic effect. In other embodiments, the combination of the first and the second active agents produces a synergistic therapeutic effect.

In some embodiments, the first active agent is a therapeutic agent and the second active agent is a targeting agent that can bind a specific surface receptor or ligand and can thus target the system to a specific brain region or to a particular cell population within the brain, leading to enhanced and focused treatment. In some related embodiments, said second active agent further has a therapeutic activity against a brain related disease or disorder.

In some embodiments, at least one of the first and the second active agents is a molecule having intracellular targeting capabilities, i.e., a molecule that targets an intracellular macromolecule. In some related embodiment, said molecule is conjugated to the core particle through a cleavable linker.

It is known that complex diseases are often multifactorial and involve redundant or synergistic action of disease mediators or upregulation of different receptors, including crosstalk between their signaling networks (Kontermann, R. In: MAbs. Taylor & Francis, 2012. p. 182-197). Consequently, blockade of multiple, different pathological factors and pathways may result in a significantly improved therapeutic efficacy. This result can be achieved by combining different drugs, or use of the dual targeting strategies.

In some embodiments, both the first and the second active agents can bind a specific surface receptor or ligand. Thus, in some embodiments, the multifunctional system of the invention combines specificities of two different active agents, e.g., antibodies, in a single system, enabling to simultaneously interfere with different surface receptors or ligands within the brain. Without wishing to be bound by any theory or mechanism of action, it is hypothesized that dual-targeted particles (e.g., dual-antibody particles) can bring different targets in the brain into close proximity, either to support protein complex formation on one cell, or to trigger contacts between cells. In some embodiments, the first and the second active agents are antibodies wherein at least one of said first and second active agents is a bispecific antibody. Thus, in some embodiments, the multifunctional system of the invention enables to simultaneously interfere with more than two targets. In some related embodiments, at least one of the first and the second active agents further has a therapeutic activity against a brain related disease or disorder. In some embodiments, both the first and the second active agents further have a therapeutic activity against a brain related disease or disorder.

In some embodiments, the first active agent is an antibody and the second active agent is selected from the group consisting of an antibody, a peptide, a small molecule, an oligonucleotide, an antisense RNA, and any fragment or combination thereof. In a related embodiment, the first active agent is an antibody and the second active agent is Fas ligans (FasL) or another death inducing receptor ligand. In some embodiments, the first active agent is an antibody and the second active agent is selected from the group consisting of a peptide, a small molecule, an oligonucleotide, an antisense RNA, and any fragment or combination thereof. Each possibility represents a separate embodiment of the present invention. In some embodiments, the first active agent is an antibody and the second active agent is a small molecule.

In some embodiments, each one of the first active agent and the second active agents is an antibody or a fragment thereof. In some embodiments, each one of the first active agent and the second active agents is an antibody or an active fragment thereof. In some embodiments, each one of the first active agent and the second active agents is an antibody or an antigen binding fragment thereof. In some related embodiments, the first active agent and the second active agent comprise different antibodies. In other related embodiments, the first active agent and the second active agent comprise or consists of different fragments of the same antibody. For example, in some embodiments, the first active agent comprises or consists of the Fab region of an antibody and the second active agent comprises or consists of the Fc region of the same antibody. In other embodiments, the first active agent comprises or consists of a whole antibody (e.g., IgG) and the second active agent comprises or consists of a fragment of the same antibody. For example, in some embodiments, the first active agent comprises or consists of a whole antibody (e.g., IgG) and the second active agent comprises or consists of an Fc region of the same antibody.

In some embodiments, the first active agent is a peptide and the second active agent is selected from the group consisting of an antibody, a peptide, a small molecule, an oligonucleotide, an antisense RNA, and any fragment or combination thereof. Each possibility represents a separate embodiment of the present invention.

In some embodiments, the first active agent is a small molecule and the second active agent is selected from the group consisting of an antibody, a peptide, a small molecule, an oligonucleotide, an antisense RNA, and any fragment or combination thereof. Each possibility represents a separate embodiment of the present invention.

In some embodiments, the first active agent is an oligonucleotide and the second active agent is selected from the group consisting of an antibody, a peptide, a small molecule, an oligonucleotide, an antisense RNA, and any fragment or combination thereof. Each possibility represents a separate embodiment of the present invention.

In some embodiments, the first active agent is an antisense RNA and the second active agent is selected from the group consisting of an antibody, a peptide, a small molecule, an oligonucleotide, an antisense RNA, and any fragment or combination thereof. Each possibility represents a separate embodiment of the present invention.

According to some embodiments, the core particle is a gold nanoparticle. According to some embodiments, the first linear polymeric linker is a thiolated PEG3500 acid or thiolated PEG35000 amine. According to some embodiments, the second linear polymeric linker is a thiolated PEG3500 acid or thiolated PEG3500 amine. According to some embodiments, the third linear polymeric linker is a thiolated PEG5000 acid or thiolated PEG5000 amine. According to some embodiments, the brain-internalizing transporter moiety is insulin.

According to some embodiments, the core particle is a gold nanoparticle. According to some embodiments, the first linear polymeric linker is a thiolated PEG3500 acid or thiolated PEG35000 amine. According to some embodiments, the second linear polymeric linker is a thiolated PEG1000 acid or thiolated PEG1000 amine. According to some embodiments, the third linear polymeric linker is a thiolated PEG5000 acid or thiolated PEG5000 amine. According to some embodiments, the brain-internalizing transporter moiety is insulin.

In some embodiments, the multifunctional particle further comprises at least one additional active agent that is attached to the core particle through an additional polymeric linker. The different possibilities of the at least one additional active agent and the respective polymeric linker are similar to those described above for the first and the second active agents, and the first and second polymeric linker.

In some embodiments, the invention provides a plurality of multifunctional particles as described above in all embodiments thereof.

### Preparation Processes

According to another aspect, there is provided a process for preparation of the multifunctional particle of the invention, in all embodiments thereof as described above, the process comprising the steps of:
a) partially coating a surface of a core particle with a first polymeric linker followed by conjugating the first polymeric linker to a first active agent;
b) partially coating the surface of the core particle with a second polymeric linker followed by conjugating the second polymeric linker to a second active agent;
c) partially coating the surface of the core particle with a third polymeric linker followed by conjugating the third polymeric linker to the brain internalizing transporter moiety, wherein steps (a), (b) and (c) can be performed in any order.

The term “partially coating”, as used herein, refers to conjugating a plurality of the respective polymeric linkers to the surface of a particle, such that the plurality of linkers partly covers the surface of the particle at a density level below the saturation level of the naked particle.

Any method known in the art can be used for determining the amount of polymer required for achieving full-density (i.e., 100%) coating of a particle, and accordingly the amount needed for partial coating. For example, adding different amounts of polymer to the particle solution and measuring the concentration of the free polymer in supernatants after centrifugation is a widely used method. Alternatively, any characterization method that is sensitive to changes in coating density can be used, such as zeta potential and DLS. Furthermore, theoretical calculations can be performed to determine the amount of polymer needed to achieve complete coating according to the surface area of the particle. For example, it was previously shown that a thiol-PEG molecule occupies a footprint area 0.35 nm² on gold nanoparticle surface (Qian, Ximei, et al. Nature biotechnology 26.1 (2008): 83-90). Accordingly, the amount of a thiol-PEG linker required to cover 100% of the surface of a gold nanoparticle (GNP) can be calculated based on the mean diameter of the GNP.

In some embodiments, each one of the first polymeric linker, the second polymeric linker and the third polymeric linker is added in an amount suitable for covering between 5-70%, 5-60%, 5-40%, 8-60%, 10-60%, 10-55%, 10-50%, 10-40%, 10-30%, 10-25%, 10-20%, 15-60%, 15-55%, 15-50%, 15-45%, 15-40%, 15-30%, 15-25%, 15-20%, 2-10%, 2-20%, 2-50%, 2-60%, 2-70%, 5-10%, 5-20%, 5-70%, 10-20%, 10-50%, 10-70%, 20-50%, 20-40%, 30-50%, 30-60% or30-70% of the surface of the core particle. Each possibility represents a separate embodiment of the present invention.

In some embodiments, step (a) comprises coating between 5-70%, 5-60%, 5-40%, 8-60%, 10-60%, 10-55%, 10-50%, 10-40%, 10-30%, 10-25%, 10-20%, 15-60%, 15-55%, 15-50%, 15-45%, 15-40%, 15-30%, 15-25%, 15-20%, 2-10%, 2-20%, 2-50%, 2-60%, 2-70%, 5-10%, 5-20%, 5-70%, 10-20%, 10-50%, 10-70%, 20-50%, 20-40%, 30-50%, 30-60%, 30-70%, 50-60% or 50-70% of the surface of the core particle. Each possibility represents a separate embodiment of the present invention.

In some embodiments, step (b) comprises coating between 5-70%, 5-60%, 5-40%, 8-60%, 10-60%, 10-55%, 10-50%, 10-40%, 10-30%, 10-25%, 10-20%, 15-60%, 15-55%, 15-50%, 15-45%, 15-40%, 15-30%, 15-25%, 15-20%, 2-10%, 2-20%, 2-50%, 2-60%, 2-70%, 5-10%, 5-20%, 5-70%, 10-20%, 10-50%, 10-70%, 20-50%, 20-40%, 30-50%, 30-60%, 30-70%, 50-60% or 50-70% of the surface of the core particle. Each possibility represents a separate embodiment of the present invention.

In some embodiments, step (c) comprises coating between 5-70%, 5-60%, 5-40%, 8-60%, 10-60%, 10-55%, 10-50%, 10-40%, 10-30%, 10-25%, 10-20%, 15-60%, 15-55%, 15-50%, 15-45%, 15-40%, 15-30%, 15-25%, 15-20%, 2-10%, 2-20%, 2-50%, 2-60%, 2-70%, 5-10%, 5-20%, 5-70%, 10-20%, 10-50%, 10-70%, 20-50%, 20-40%, 30-50%, 30-60%, 30-70%, 50-60% or 50-70% of the surface of the core particle. Each possibility represents a separate embodiment of the present invention.

In some embodiments, steps (a)-(c) are performed sequentially, in any order. A person skilled in the art would be able to determine the optimal order of the steps according to different parameters, e.g., the core particle type, the specific polymeric linkers, the active agents used, the brain internalizing transporter moiety and the like. In some embodiments, the process further comprises centrifugation after each one of steps (a), (b) and (c).

In some embodiments, the first polymeric linker and the second polymeric linker are identical. In some related embodiments, steps (a) and (b) are performed simultaneously, by partially coating the surface of the core particle with the first and second polymeric linkers together, and then conjugating the first and the second active agents to the polymeric linkers. In some related embodiments, the step of partially coating the surface of the core particle with the first and second polymeric linkers together, comprises coating between 10-70%, 10-60%, 10-40%, 10-60%, 10-60%, 10-55%, 10-50%, 10-45%, 10-40%, 10-30%, 10-25%, 10-20%, 15-60%, 15-55%, 15-50%, 15-45%, 15-40%, 15-30%, 15-25%, 15-20%, 10-20%, 10-50%, 10-70%, 20-50%, 20-40%, 30-50%, 30-60%, 30-70%, 50-60% or 50-70% of the surface of the core particle. Each possibility represents a separate embodiment of the present invention. In further related embodiments, conjugating the first and the second active agents to the polymeric linkers comprises adding a mixture of the first and the second active agents in the desired molar ratio, to the particle solution.

In some embodiments, the process further comprises partially coating the surface of the core particle with a fourth polymeric linker. In some embodiments, the fourth polymeric linker is a monofunctional linker.

According to related embodiments, there is provided a process for preparation of a multifunctional particle, the process comprising the steps of:
a) partially coating a surface of a core particle with a first polymeric linker followed by conjugating the first polymeric linker to a first active agent;
b) partially coating the surface of the core particle with a second polymeric linker followed by conjugating the second polymeric linker to a second active agent;
c) partially coating the surface of the core particle with a third polymeric linker followed by conjugating the third polymeric linker to the brain internalizing transporter moiety;
and
d) partially coating the surface of the core particle with a fourth polymeric linker, wherein the fourth polymeric linker is a monofunctional linker serving as a capping moiety, wherein steps (a), (b), (c) and (d) can be performed in any order.

In some embodiments, the particle is a gold nanoparticle (GNP) and the process comprises the sequential steps of: (a) reduction of HAuCl₄; (b) simultaneous incubation of the reduced GNPs with one mono-functional linker and two different heterofunctional linkers; (c) activation the GNPs to obtain free COOH groups; (d) conjugation of the transporter or other moiety; (d) conjugation of the two different bioactive molecules by incubating with a solution comprising their mixture.

In some embodiments, the mono-functional linker is mPEG-SH. According to a specific embodiment, the mono-functional linker is mPEG5000-SH or mPEG6000-SH and it is added to cover about 80-90% of particle surface.

In some embodiments, the heterofunctional linker is COOH-PEG-SH. According to some embodiments, one heterofunctional linker is COOH-PEG5000-SH and it is added in a concentration to cover about 15% of particle surface. According to some embodiments, the other heterofunctional linker is COOH-PEG3500-SH and it is added in a concentration to cover about 5% of particle surface.

In some embodiments, activation of the GNPs is performed by mixing the GNPs with (1-ethyl-3-(3-dimethylaminopropyl) carbodiimide HCl (EDC).

In some embodiments, the transporter is insulin, and its conjugation is performed by incubating for 1-5 hours with the activated GNPs, at a concentration of about 50-500 IU/ml.

In some embodiments, the two bioactive molecules are incubated overnight at a concentration of 1-50 mg/ml, with activated GNPs.

Analysis of the GNPs is performed following each step using methods known in the art, for example using Dynamic Light Scattering (DLS).

In some embodiments, quantification of the bioactive molecules and the transporter (e.g., insulin) attached to the PEG groups on the GNPs is performed by enzyme-linked immunosorbent assay (ELISA) of the supernatants containing the unbound proteins left after precipitation by centrifugation of the GNPs.

The core particle, the first polymeric linker, the second polymeric linker, the third polymeric linker, the fourth polymeric linker, the brain-internalizing transporter moiety, and the first and second active agents suitable for use in the preparation process are those described hereinabove in connection with the various aspects and embodiments of the co-delivery system.

### Pharmaceutical Compositions

In yet another aspect, there is provided a pharmaceutical composition comprising the multifunctional particle according to the various embodiments described hereinabove and a pharmaceutically acceptable carrier. In some embodiments, the pharmaceutical composition comprises a plurality of multifunctional particles according to the various embodiments described hereinabove, and a pharmaceutically acceptable carrier.

As used herein, a “pharmaceutically acceptable formulation,” “pharmaceutical composition” or “pharmaceutically acceptable composition” may include any of a number of carriers such as solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's, 1990). Pharmaceutical compositions containing the presently described particles as the active ingredient can be prepared according to conventional pharmaceutical compounding techniques. See, for example, Remington's Pharmaceutical Sciences, 18th Ed., Mack Publishing Co., Easton, Pa. (1990). See also, Remington: The Science and Practice of Pharmacy, 21st Ed., Lippincott Williams & Wilkins, Philadelphia, Pa. (2005).

A composition may comprise different types of carriers depending on whether it is to be administered in solid, liquid or aerosol form, and whether it needs be sterile for such routes of administration as injection. A person of ordinary skill in the art would be familiar with techniques for generating sterile solutions for injection or application by any other route. Sterile injectable solutions are prepared by incorporating the active compounds in the required amount in an appropriate solvent with various other ingredients familiar to a person of skill in the art.

The carrier may comprise, in total, from about 0.1% to about 99.99999% by weight of the pharmaceutical compositions presented herein.

According to some embodiments, the pharmaceutical composition is formulated for systemic administration. According to some embodiments, the pharmaceutical composition is formulated for systemic administration selected from intravenous and intranasal administration. According to some embodiments, the pharmaceutical composition is formulated for intravenous administration. According to some embodiments, the pharmaceutical composition is formulated for intranasal administration. According to some embodiments, the pharmaceutical composition is formulated for intrathecal administration.

The compositions contemplated herein may take the form of solutions, suspensions, emulsions, aerosols, combinations thereof, or any other pharmaceutical acceptable composition as would commonly be known in the art.

In some embodiments, the carrier is a solvent. For a non-limiting example, the composition may be disposed in the solvent. Such a solvent includes any suitable solvent known in the art such as water, saline, phosphate-buffered saline.

The formulation of the composition may vary depending upon the route of administration. For parenteral administration in an aqueous solution, for example, the solution should be suitably buffered if necessary and the liquid diluent first rendered isotonic with sufficient saline or glucose. Sterile aqueous media which can be employed will be known to those of skill in the art in light of the present disclosure.

Supplementary active ingredients can also be incorporated into the compositions. For human administration, preparations should meet sterility and general safety and purity standards as required by FDA Office of Biologics standards. Administration may be by any known route.

In certain embodiments, a pharmaceutical composition includes an amount of equivalent to at least about 0.001 g to about 1 g of the particle disclosed herein per kilogram of a subject. In certain embodiments, a pharmaceutical composition includes at least about 0.001 g to about 0.5 g of the particle disclosed herein per kilogram of a subject.

The pharmaceutical composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof. The composition must be stable under the conditions of manufacture and storage, and preserved against the contaminating action of microorganisms, such as bacteria and fungi. It will be appreciated that exotoxin contamination should be kept minimally at a safe level, for example, less than 0.5 ng/mg protein.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. In many cases, it will be preferable to include isotonic agents, such as, for example, sugars, sodium chloride or combinations thereof.

In other embodiments, a nasal solutions or sprays, aerosols or inhalants may be used. Nasal solutions are usually aqueous solutions designed to be administered to the nasal passages in drops or sprays.

Solid compositions for oral administration are also contemplated. In these embodiments, the solid composition may comprise, for example, solutions, suspensions, emulsions, tablets, pills, capsules, sustained release formulations, buccal compositions, troches, elixirs, suspensions, syrups, or combinations thereof.

Sterile injectable solutions are prepared by incorporating the active compounds (e.g., nanoparticles) in the required amount in the appropriate solvent with various other ingredients enumerated above. The liquid medium should be suitably buffered if necessary and the liquid diluent first rendered isotonic prior to injection with sufficient saline or glucose.

The dose can be repeated as needed as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art.

### Therapeutic and Diagnostic Use of the Composition

According to some aspects, there is provided a pharmaceutical composition comprising the multifunctional particle of the invention, for use in the co-delivery of a first and a second active agents to the brain of a subject in need thereof.

According to other aspects, the present invention provides a method for a synchronized delivery of a first and a second active agents to the brain of a subject in need thereof, the method comprises administering to the subject a pharmaceutical composition comprising the multifunctional particle described above in all embodiments thereof.

According to some embodiments, the pharmaceutical composition is for use in treating a brain-related disease or disorder in need thereof. According to some embodiments, the pharmaceutical composition is for use in preventing a brain-related disease or disorder in a subject in need thereof. According to some embodiments, the pharmaceutical composition is for use in monitoring a brain-related disease or disorder in a subject in need thereof.

According to some aspects and embodiments, there is provided a method for treating a brain related disease or disorder in a subject in need thereof, the method comprising administering the pharmaceutical composition of the invention to the subject.

According to some aspects and embodiments, there is provided a method for preventing a brain related disease or disorder in a subject, the method comprising administering the pharmaceutical composition of the invention to the subject.

According to some aspects and embodiments, there is provided a method for monitoring a brain related disease or disorder in a subject in need thereof, the method comprising administering the pharmaceutical composition of the invention to the subject and imaging the brain of the subject. In some related embodiments, the pharmaceutical composition comprises the multifunctional particle described above wherein the core particle is a gold nanoparticle and the imaging is performed using CT to enable detection of the multifunctional particle within the brain. In other embodiments, the pharmaceutical composition comprises the multifunctional particle described above wherein at least one active agent is a labeling molecule, e.g., a fluorescent or radioactive molecule which allows detection by a suitable imaging modality. In some embodiments, the method for monitoring the brain related disease or disorder comprises repeated administrations and/or repeated imaging sessions.

As used herein, the term “brain-related disease or disorder” refers to any disease or disorder that causes malfunction of the brain or any cell thereof. Non-limiting examples of brain-related diseases and disorders are neurodegenerative disorders such as Parkinson's disease, Alzheimer's disease, Huntington's disease and dementia; neuromuscular diseases such as amyotrophic lateral sclerosis (ALS) and motor neuron disease; neurodevelopmental diseases such as autism spectrum disorders and attention deficit hyperactivity disorder (ADHD); autoimmune brain-related diseases such as multiple sclerosis (MS); neuropsychiatric disorders such as schizophrenia, addiction such as drug addiction and smoking addiction, eating disorders, obsessive-compulsive disorder, various forms of depression, anxiety disorders, cognitive disorders and affective disorders; seizure disorders such as epilepsy; pain disorders such as migraine; cerebrovascular disorders including traumatic brain injury and stroke; brain-related cancers such as brain and nerve tumors, brain metastasis, glioma, glioblastoma (GBM), and gliosarcoma (GS); neurogenetic diseases such as Huntington's disease, Kennedy's disease, metabolic disorders, lysosomal storage disorders and Duchenne; and neuroinfectious diseases.

In some embodiments, the disease is a central nervous system disease. According to some embodiments, the disorder is a brain disorder.

In some embodiments, the pharmaceutical composition is for use in the treatment of a brain-related disease or disorders. In some embodiments, the brain-related disease or disorder is selected from the group consisting of a brain-related cancer, a neurodegenerative disorder, a neuromuscular disease, a neurodevelopmental disease, an autoimmune brain-related disease, a neuropsychiatric disorder, a seizure disorder, a pain disorders, a cerebrovascular disorder, a neurogenetic disease and a neuroinfectious disease.

In some embodiments, the brain-related disease is a brain-related cancer. The term “brain related cancer” as used herein encompasses both primary brain tumors (i.e., primary brain cancer) and metastatic brain tumors (i.e., secondary brain cancer). In some embodiments, the brain related cancer is selected from but not limited to the group consisting of brain and nerve tumors, brain metastasis, glioma, glioblastoma (GBM), and gliosarcoma (GS). In some embodiments, the brain-related disease is a neurodegenerative disorder. In some embodiments, the neurodegenerative disorder is selected from the group consisting of Parkinson's disease, Alzheimer's disease, Huntington's disease and dementia. In some embodiments, the brain-related disease is a neuromuscular disease. In some embodiments, the neuromuscular disease is selected from the group consisting of amyotrophic lateral sclerosis (ALS) and motor neuron disease. In some embodiments, the brain-related disease is a neurodevelopmental disease. In some embodiments, the neurodevelopmental disease is selected from the group consisting of autism spectrum disorders and attention deficit hyperactivity disorder (ADHD). In some embodiments, the brain-related disease is multiple sclerosis (MS). In some embodiments, the brain-related disease is a neuropsychiatric disorder. In some embodiments, the neuropsychiatric disorder is selected from the group consisting of schizophrenia, addiction, such as drug addiction and smoking addiction, eating disorders, obsessive-compulsive disorder, various forms of depression, anxiety disorders, cognitive disorders and affective disorders. In some embodiments, the brain-related disease is a seizure disorder. In some embodiments, the seizure disorder is epilepsy. In some embodiments, the brain-related disease is a pain disorder. In some embodiments, the brain-related disease is a cerebrovascular disorder. In some embodiments, the cerebrovascular disorder is selected from traumatic brain injury and stroke. In some embodiments, the brain-related disease is a neurogenetic disease. In some embodiments, the neurogenetic disease is selected from the group consisting of Huntington's disease, Kennedy's disease, metabolic disorders, lysosomal storage disorders and Duchenne. In some embodiments, the brain-related disease is a neuroinfectious disease.

In some embodiments, the brain-related disease is Alzheimer's disease. In some embodiments, the brain-related disease is Parkinson's disease. According to some embodiments, the brain-related disease is Huntington's disease, spinocerebellar ataxia, amyotrophic lateral sclerosis, Friedreich's ataxia, motor neuron disease (Lou Gehrig's disease) or spinal muscular atrophy. According to some embodiments, the brain-related disease is a prion disease.

As used herein, the term “subject” refers to any animal (e.g., a mammal), including, but not limited to, humans, non-human primates, rodents, and the like (e.g., which is to be the recipient of a particular treatment). Typically, the terms “subject” and “patient” are used interchangeably, unless indicated otherwise herein.

In some embodiments, the subject is a human subject. In some embodiments, the subject is at risk of being afflicted with a brain-related disease, a disorder, or a medical condition. In some embodiments, the subject is diagnosed with a brain-related disease, a disorder, or a medical condition. In some embodiments, the subject is diagnosed with a brain-related genetic disorder. In some embodiments, the subject is diagnosed with a brain-related cancer. In some embodiments, the subject is at risk of being afflicted with a neurodegenerative disease. In some embodiments, the subject is diagnosed with a neurodegenerative disease. In some embodiments, the subject is diagnosed with Alzheimer's disease. In some embodiments, the subject is diagnosed with Parkinson's disease.

As used herein, a subject at risk of being afflicted with a disease, a disorder, or a medical condition, is a subject that presents one or more signs or symptoms indicative of a disease, a disorder, or a medical condition or is being screened for a disease, a disorder, or a medical condition (e.g., during a routine physical). A subject at risk of being afflicted with a disease, a disorder, or a medical condition, may also have one or more risk factors. A subject at risk of being afflicted with a disease, a disorder, or a medical condition encompasses an individual that has not been previously tested for the disease, disorder, or medical condition. However, a subject at risk of being afflicted with a disease, a disorder, or a medical condition, also encompasses an individual who has received a preliminary diagnosis but for whom a confirmatory test (e.g., biopsy and/or histology) has not been done or for whom the stage of the disease, disorder, or medical condition is not known. The term further includes people who once had the disease, disorder, or medical condition (e.g., an individual in remission).

A subject at risk of being afflicted with a brain-related disease, disorder, or medical condition may be diagnosed as having or alternatively found not to have the brain-related disease, disorder, or medical condition.

As used herein, a subject diagnosed with a brain-related disease, disorder, or medical condition, may be diagnosed using any suitable method, including but not limited to, biopsy, x-ray, blood test, and the diagnostic methods of the present invention. A “preliminary diagnosis” is one based only on visual (e.g., CT scan or the presence of a lump) and antigen tests.

As used herein, the terms “treatment”, “treating”, or “ameliorating” of a disease, disorder, or condition, refer to alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

In some embodiments, the method further comprises a step of imaging the brain region of the subject. In some embodiments, the imaging is performed using an imaging system selected from the group consisting of: computed tomography imaging (CT), X-ray imaging, magnetic resonance imaging (MRI), positron emission tomography (PET), single-photon emission computed tomography (SPECT), ultrasound (US), and any combination thereof.

In some embodiments, the imaging is performed to evaluate accumulation of the co-delivery system in the brain of said subject.

In some embodiments, the subject is afflicted with a brain-related disease or disorder, and the imaging is performed for determining the stage of the disease or disorder. In some embodiments, the subject afflicted with a brain-related disease or disorder was treated with a drug and the imaging method is used for follow-up of the treatment.

In some embodiments, the imaging step is performed 0.5 to 96 hours post the administering step. In some embodiments, the imaging step is performed 0.5 to 48 hours post the administering step. In some embodiments, the imaging step is performed 0.5 to 24 hours post the administering step. In some embodiments, the imaging step is performed 0.5 to 12 hours post the administering step. In some embodiments, the imaging step is performed 1 to 12 hours post the administering step. In some embodiments, the imaging step is performed 1 to 6 hours post the administering step. In some embodiments, the imaging step is performed within 96 hours from the administering step. In some embodiments, the imaging step is performed within 48 hours from the administering step. In some embodiments, the imaging step is performed within 24 hours from the administering step. In some embodiments, the imaging step is performed within 12 hours from the administering step. In some embodiments, the imaging step is performed within 6 hours from the administering step.

Administering the composition to the subject can be done by using any method known to those of ordinary skill in the art. The mode of administering may vary based on the application. For example, the mode of administration may vary depending on the particular cell, brain region, or subject to be imaged. For example, administering the composition may be done intravenously, intracerebrally, intracranially, intrathecally, intracerebroventricular, into the substantia nigra or the region of the substantia nigra, intradermally, intraarterially, intraperitoneally, intralesionally, intratracheally, intranasally, intramuscularly, intraperitoneally, subcutaneously, orally, topically, locally, by inhalation (e.g., aerosol inhalation), by injection, by infusion, by subarachnoid infusion, by transmucosal infusion, by intracarotid infusion, by continuous infusion, by localized perfusion bathing target cells directly, via a catheter, via a lavage, or by other method or any combination of the forgoing as would be known to one of ordinary skill in the art.

In some embodiments, the pharmaceutical composition is administered to the subject by a systemic administration route. In some embodiments, the systemic administration is selected from an intravenous (IV) administration and an intranasal (IN) administration. In some embodiments, the pharmaceutical composition is administered to the subject by intrathecal (IT) administration. In some embodiments, the particle is administered intravenously. In some embodiments, the particle is administered intranasally.

An effective amount of the pharmaceutical composition is determined based on the intended goal and the subject to be treated. The amount to be administered may also vary based on the particular route of administration to be used. The composition is preferably administered in a safe and effective amount. As used herein, the term “safe and effective amount” refers to the quantity of a composition which is sufficient for the intended goal without undue adverse side effects (such as toxicity, irritation, or allergic response).

In some embodiments, the method further comprises using an additional therapy. In some embodiments, in particular wherein the brain-related disease is a brain-related cancer, the additional therapy is selected from but not limited to, surgery, radiation therapy and chemotherapy. In specific embodiments, the additional therapy is radiation therapy.

In some embodiments, the core particle is a radiosensitizer and the method of treatment the brain related cancer further comprises a step of directing an ionizing irradiation to the tumor cells (in which the particles accumulate) thereby obtaining locally enhanced radiation therapy within the tumor cells. In some embodiments, the composition is used for thermal ablation of tumor cells in which the composition accumulates using Infra-Red waves, without causing damage to surrounding normal tissues or substantial toxicity to the subject. As used herein, “ablation” refers to the destruction of cells. Methods for irradiating a tissue comprising metal particles for enhancing effects of radiation therapy, are known in the art.

### Kits

In some embodiments, the invention provides kits comprising one or more compositions disclosed herein. In some embodiments, the invention provides kits useful for methods disclosed herein. For example, a kit may include a container having a sterile reservoir that houses any composition disclosed herein. In some embodiments, the kit further includes instructions. For example, a kit may include the instructions for administering the composition to a subject (e.g., indication, dosage, methods etc.). In yet another example the kit may include instructions regarding application of the compositions and methods of the invention to imaging systems e.g., computed tomography (CT), ultrasound (US), magnetic resonance imaging (MRI).

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Any concentration ranges, percentage range, or ratio range recited herein are to be understood to include concentrations, percentages or ratios of any integer within that range and fractions thereof, such as one tenth and one hundredth of an integer, unless otherwise indicated.

Any number range recited herein relating to any physical feature, such as polymer subunits, size or length, are to be understood to include any integer within the recited range, unless otherwise indicated.

As used herein, the term “about” when combined with a value refers to plus and minus 10% of the reference value. For example, a molecular weight of about 1000 Da refers to a molecular weight of 1000 Da+−100 Da.

It is noted that as used herein and in the appended claims, the singular forms “a,” “an,” and “the” include plural referents unless the context clearly dictates otherwise. Thus, for example, reference to “a particle” includes a plurality of such particles and reference to “the particle” includes reference to one or more particles. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “plurality” means “two or more”, unless expressly specified otherwise.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein. The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art. Accordingly, it is intended to embrace all such modifications and variations that fall within the spirit and broad scope of the appended claims.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Strategies for Protein Purification and Characterization-A Laboratory Course Manual” CSHL Press (1996); all of which are incorporated by reference. Other general references are provided throughout this document.

### Example 1: Multifunctional Gold Nanoparticles (GNPs) Co-Deliver Two Antibodies into the Brain

FIG. 1 shows a schematic illustration of a non-limiting exemplary multifunctional particle, showing a gold nanoparticle (GNP;1) bound to: (i) a first polymeric linker (2) which is conjugated to a first antibody (3); (ii) a second polymeric linker (4) which is conjugated to a second antibody (5); (iii) a third polymeric linker (6) which is conjugated to a brain internalizing transporter moiety (e.g., insulin; 7); and (iv) a monofunctional polymer moiety (8).

### Preparation and Characterization of GNPs Conjugated to Anti-IgG1, Anti-Iba1 and Insulin (IgG1 &Iba1 &Ins-GNPs)

GNP synthesis: 20 nm spherical GNPs were prepared by citrate reduction of HAuCl₄. A total of 414 μl of 50% w/v of HAuCl₄ solution in 200 ml distilled water was boiled in an oil bath on a heating plate while being stirred. After boiling, 4.04 ml of a 10% sodium citrate solution were added and the mixture was stirred while boiling for another 10 minutes. The solution was removed from the plate and after cooling to room temperature, the solution was centrifuged until precipitation of the nanoparticles.

Conjugation of PEG5000 to the GNPs: GNPs were first partially coated (60% of particle surface) with mPEG-SH (˜5 kDa; 40% of particle surface) and a heterofunctional HS-PEG-COOH (˜5 kDa; 20% of particle surface). The amount of mPEG-SH and HS-PEG-COOH required for the partial coating was derived from theoretical calculations based on the finding that thiol-PEG molecule occupies a footprint area 0.35 nm² on gold nanoparticle surface (Qian, Ximei, et al. Nature biotechnology 26.1 (2008): 83-90). Conjugation was performed by adding a mixture of HS-PEG-COOH (193 μl, 50 mg/ml) and mPEG-SH (387 μl, 50 mg/ml) to the GNP solution and mixing for two hours. The solution was then ultra-centrifuged at 15,000 RPM for 20 minutes and then again at 20,000 RPM for 15 minutes. The precipitate, containing the PEG-coated GNPs (total 60% coating) was transferred to a vial.

Conjugation of insulin: In order to facilitate the transport of the multifunctional GNP through the BBB, insulin was covalently conjugated to the carboxylic group of the HS-PEG-COOH by addition of excess amount of insulin on ice together with EDC (1-ethyl-3-(3-dimethylaminopropyl) carbodiimide HCl) and NHS (N-hydroxysulfosuccinimide sodium salt) followed by mixing for two hours. Then, the solution was centrifuged twice at 15,000 RPM for 30 minutes (maintained at a cool temperature) and the lower phase, containing the Ins-PEG-GNPs was transferred into a vial.

Conjugation of PEG3500 to the GNPs: In order to further conjugate antibodies to the GNPs, 271 μl of HS-PEG-COOH (˜3.5 kDa) solution (50 mg/ml) were added to the partially coated GNPs to coat the remaining 40% of particle surface. The solution was then mixed for two hours at 4° C. followed by repetitive centrifugation at 15,000 RPM for 30 minutes.

Conjugation of anti-IgG1 and anti-Iba1: Fluorescently labeled anti-Iba1 and Fluorescently labeled anti-IgG1 were covalently conjugated to free carboxylic groups of the HS-PEG-COOH (˜3.5 kDa) by addition of 1:1 molar mixture of fluorescently labeled anti-Iba1 (Ab195032-Rb Mono & Hu IBA-1-647) and fluorescently labeled anti-IgG1 (mouse Monoclonal IgG1 Alexa Fluor 488 Isotype Control Clone 11711) together with EDC and NHS. The solution was then stirred for 2 hours at 4° C. followed by centrifugation to remove unbound antibodies, until a final concentration of 25 mg/ml Au was reached.

To confirm the chemical conjugation of the antibodies, the multifunctional dual antibody nanoparticles were imaged using a fluorescent microscope. Fluorescent signals of both anti-IgG1 and anti-Iba1 were detected, indicating a successful chemical conjugation.

For control experiments, similar particles were prepared with only one conjugated antibody, i.e., anti-IgG1 or anti-Iba1. % coverage of the different coating molecules in the control particles: 20% PEG5000 (conjugated to insulin), 20% PEG3500 (conjugated to the respective antibody) and 60% mPEG5000.

Delivery of the Dual-Antibody Conjugated GNPs into Mice Brain

Male BALB/c mice were divided into five treatment groups:
Group 1: Saline injected (Naïve, control; n=2)
Group 2: anti-IgG1 & insulin-conjugated GNPs (IgG1&Ins-GNPs; n=2)
Group 3: anti-Iba1 & insulin-conjugated GNPs (Iba1&Ins-GNPs; n=2)
Group 4: Multifuctional dual-antibody GNPs (IgG1&Iba1&Ins-GNPs; n=2)
Group 5: Free anti-IgG1 and anti-Iba1 antibodies (Free Abs; n=2)

Mice were injected intravenously with IgG1&Ins-GNPs (200 μl, 25 mg/ml), Iba1&Ins-GNPs (200 μl of 25 mg/ml), IgG1&Iba1&Ins-GNPs (200 μl of 25 mg/ml), or with equivalent amounts of free fluorescently labeled antibodies (200 μl solution containing 0.2 mg of anti-IgG1 and 0.25 mg of anti-Iba1). Eight hours post injection, mice underwent perfusion to remove all the particles/antibodies residing within the blood vessels. Then, mice brains were extracted and analyzed using ICP-OES (Groups 2, 3 and 4; n=2 per group) or by histology assessment (Groups 1, 4 and 5; n=2 per group).

FIG. 2 shows the amounts of gold within mice brains as measured by ICP-OES. It can be seen that following IV injection of IgG1&Ins-GNPs, Iba1&Ins-GNPs or IgG1&Iba1 &Ins-GNPs, significant amounts of gold have been found in mice brains indicating successful penetration through the BBB into the brain. Interestingly, penetration of the multifunctional dual antibody GNPs (IgG1&Iba1 &Ins-GNPs) was even higher that of Iba1 &Ins-GNPs.

For histology assessment of mice brains (groups 1, 4 and 5), 7 μm brain frozen sections of cerebral cortex and medulla were prepared and immuno-stained (IHC-F double staining). Fluorescence antibodies signals were detected and photos were taken using confocal microscope. All photos for each antibody were taken under the same exposure conditions.

FIGS. 3 and 4 show representative immunocytochemistry-fluorescence (IHC-F) images of a cerebral cortex section (FIG. 3) and a medulla section (FIG. 4). Remarkably, whereas no antibody signal was observed in brain sections of mice that were administered with the free antibodies (Group 5), both anti-IgG1 and anti-Iba1 signals were detected in the brains of mice that received multifunctional brain targeted GNPs conjugated with these antibodies (Group 4). Moreover, the merged images show co-localization of the two antibodies within the cerebral cortex and the medulla, indicating synchronized distribution of the antibodies in the brain.

### Example 2: Preparation of Multifunctional GNPs Coated with Insulin and with Two Anti Her2 Antibodies, Trastuzumab and Pertuzumab

As a non-limiting example, gold nanoparticles carrying to different anti HER2 antibodies and insulin, were produces as schematically described in FIG. 6. The exemplary particle was coated with a polymeric layer (2-3 in FIG. 6) comprising two polymeric linkers (5—S-PEG-C(O)—, ˜5 kDa and —S-PEGC(O)—, ˜3.5 kDa) the first linker was conjugated to insulin (4) and the second linker is conjugated to two different anti Her2 antibodies (5-6) Trastusumab and Pertusumab. An additional (7) polymeric moiety (—S-PEG-O—CH3 ˜6 kDa) is used as a capping to control the density of the other moieties on the particle.

### GNP Synthesis

20 nm spherical GNPs were prepared by citrate reduction of HAuCl₄. A total of 414 μl of 42.77% w/v of HAuCl₄ in double distilled water (DDW) in 200 ml DDW was boiled in an oil bath on a heating plate while being stirred. After boiling, 4.04 ml of a 10% w/v trisodium citrate in DDW were added. The solution was removed from the oil bath and left to cool under stirring room.

Conjugation of COOH-PEG5000-SH, COOH-PEG3500-SH and mPEG6000-SH to the GNPs.

GNPs were incubated with mPEG6000-SH (˜6 kDa; 80% of particle surface), a heterofunctional COOH-PEG5000-SH (˜5 kDa; 15% of particle surface) and a heterofunctional COOH-PEG3500-SH (˜5 kDa; 5% of particle surface). The amount of PEG moieties that are required for proportional coating was derived from theoretical calculations based on the findings that thiol-PEG molecule occupies a surface area of 0.35 nm² on gold nanoparticle surface (Qian, Ximei, et al. Nature biotechnology 26.1 (2008): 83-90). Conjugation was performed by adding a mixture of COOH-PEG5000-SH (127 μl, 50 mg/ml in DDW), mPEG6000-SH (809 μl, 50 mg/ml in DDW) and COOH-PEG3500-SH (30 μl, 50 mg/ml in DDW) to the GNP solution and mixing overnight. The solution was then centrifuged at 50,000 G for 20 minutes, then the precipitants were redispersed in DDW and centrifuged at 50,000 G for 20 minutes. The precipitate, containing the PEG-coated GNPs was transferred to a vial.

Activation of the GNPs was performed by mixing the GNPs with EDC (1-ethyl-3-(3-dimethylaminopropyl) carbodiimide HCl, 30 mg/ml in DDW, 100 μl) and sulfo-NHS (N-hydroxysulfosuccinimide sodium salt, 30 mg/ml in DDW, 100 μl) followed by centrifugation at 50,000 G for 20 minutes. The precipitate, containing the activated COOH groups was transferred to a vial.

Conjugation of insulin to HS-PEG5000-COOH was then performed by addition of insulin (195 IU, 100 IU/ml) to the GNPs solution for 3 h. Then a solution containing trastuzumab and pertuzumab (total 15 mg) was inserted into 2 ml borate buffer (PH 8, 0.1M) and later added to the GNP-insulin solution for the conjugation of the remaining COOH-PEG3500-SH while mixing over-night. The solution was then centrifuged at 10,000 G, 20 min. Followed by redissolving the precipitants in saline and then centrifuged at 10,000 G for 20 min.

The GNPs coated with antibodies and insulin (Abs&Ins-GNPs) were characterized following each step of preparation using Dynamic Light Scattering (DLS). The hydrodynamic size and Zeta potential of the GNPs confirmed successful coating.

The quantification of the antibodies (Abs) and insulin attached to the PEG groups on the GNPs was tested by enzyme-linked immunosorbent assay (ELISA) tested on the supernatants containing the unbound proteins left from centrifugation.

### Example 3: Preparation of and Characterization of Multifunctional GNPs Coated with Insulin, Two Multifunctional Antibodies and a Chemotherapeutic Molecule

This experiment demonstrates the conjugation to the GNPs with four linkers, as follows: SH-PEG3500-SH for a chemotherapeutic molecule, COOH-PEG5000-SH for an insulin as a transporter molecule, COOH-PEG3500-SH for the two different antibodies and mPEG (5000-6000)-SH as a spacer and capping moiety.

### GNP Synthesis

20 nm spherical GNPs were prepared by citrate reduction of HAuCl₄. A total of 414 μl of 42.77% w/v of HAuCl₄ in 200 ml double distilled water (DDW) was boiled in an oil bath on a heating plate while being stirred. After boiling, 4.04 ml of a 10% w/v trisodium citrate in DDW were added. The solution was removed from the oil bath and left to cool at room temperature while stirring.

GNPs were incubated with mPEG(5000-6000)-SH (˜5 kDa; 75% of particle surface), a heterofunctional COOH-PEG5000-SH (˜5 kDa: 15% of particle surface), a heterofunctional COOH-PEG3500-SH (˜3.5 kDa; 5% of particle surface) and an SH-PEG3500-SH (˜3.5 kDa; 5% of particle surface). The amount of PEG moieties that are required for proportional coating was derived from theoretical calculations based on the findings that thiol-PEG molecule occupies a surface area of 0.35 nm² on gold nanoparticle surface (Qian, Ximei, et al. Nature biotechnology 26.1 (2008): 83-90). Conjugation was performed by adding a mixture of COOH-PEG5000-SH (96.92 μl, 50 mg/ml in DDW), mPEG5000-SH (512.51 μl, 50 mg/ml in DDW), COOH-PEG3500-SH (23y. 14 μl, 50 mg/ml in DDW) and SH-PEG3500-SH (22.19 μl, 50 mg/ml in DDW) to the GNP solution and mixing overnight. The solution was then centrifuged at 50,000 G for 20 minutes, then the precipitants were redispersed in DDW and centrifuged at 50,000 G for 20 minutes. The precipitate, containing the PEG-coated GNPs was transferred to a vial.

Conjugation of chemotherapeutic molecule to the surface of the thiol terminated PEGs was performed by addition of the molecule to the NPs solution under stirring overnight. The following day the solution was centrifuged once at 50,000 G for 20 minutes. The precipitate was redissolved to 18 ml and transferred to a vial. The amount of chemotherapeutic molecule used in the conjugation was calculated according to the amount of particles in the reaction and is different for each molecule used.

Activation of the GNPs was performed by mixing the GNPs with EDC (1-ethyl-3-(3-dimethylaminopropyl) carbodiimide HCl, 30 mg/ml in DDW, 100 μl) and sulfo-NHS (N-hydroxysulfosuccinimide sodium salt, 30 mg/ml in DDW, 100 μl) followed by centrifugation at 50,000 G for 20 minutes. The precipitate, containing the activated COOH groups was transferred to a vial.

Conjugation of insulin to HS-PEG5000-COOH was then performed by addition of insulin (195 IU, 100 IU/ml) to the GNPs solution for 3 h. Then a solution containing the antibodies, Ab1 and Ab2 was inserted into 2 ml borate buffer (PH 8, 0.1M) and later added to the GNP-insulin solution for the conjugation of the remaining COOH-PEG3500-SH while mixing over-night. The solution was then centrifuged at 10,000 G, 20 min. Followed by redissolving the precipitants in saline and then centrifuged at 10,000 G for 20 min.

The Abs&Ins-GNPs were characterized for hydrodynamic size and zeta potential following each step of preparation using Dynamic Light Scattering (DLS). The hydrodynamic size and Zeta potential of the GNPs confirmed successful coating.

The quantification of the antibodies and insulin attached to the PEG groups on the GNPs was tested by enzyme-linked immunosorbent assay (ELISA) tested on the supernatants containing the unbound proteins left from centrifugation.

Further quantification of the antibodies and insulin is performed by stripping the coating from the GNPs surface and then quantified by HPLC using UV spectrophotometer.

The conformation of the presence of antibodies and insulin on the GNPs is tested by cell assays on BT474 Cells and by the presence of gold in mice brains.

### Example 4: Multifunctional GNPs for Co-Delivery of an Antibody and a Small Molecule Drug into the Brain

Preparation and Characterization of GNPs Conjugated to Cisplatin, Anti-IgG1 and Insulin (cisPt&IgG1&Ins-GNPs)

20 nm spherical GNPs were prepared as described in Example 1.

Conjugation of mPEG5000 and PEG1000 to the GNPs: GNPs were first partially coated (60% of particle surface) with mPEG-SH (˜5 kDa; 40% of particle surface) and a heterofunctional HS-PEG-COOH (˜1 kDa; 20% of particle surface). The amount of mPEG-SH and HS-PEG-COOH required for the partial coating was derived from theoretical calculations based on the finding that thiol-PEG molecule occupies a footprint area 0.35 nm² on gold nanoparticle surface (Qian, Ximei, et al. Nature biotechnology 26.1 (2008): 83-90). Conjugation was performed by adding a mixture of HS-PEG-COOH (40 μl, 50 mg/ml) and mPEG-SH (387 μl, 50 mg/ml) to the GNP solution and mixing for two hours. The solution was then ultra-centrifuged at 15,000 RPM for 20 minutes and then again at 20,000 RPM for 15 minutes. The precipitate, containing the PEG-coated GNPs (total 60% coating) was transferred to a vial.

Conjugation of cisplatin: Cisplatin (cisPt) was covalently conjugated to the carboxylic group of the HS-PEG-COOH (PEG1000) by addition of excess amount of cisplatin with EDC and NHS, followed by mixing for 3 hours at 4° C. Then, the solution was centrifuged at 14,000 g for 30 minutes at 4° C. and the lower phase, containing Cisplatin-GNPs was transferred into a vial.

Conjugation of PEG5000 and insulin: In order to further conjugate insulin to the GNPs, HS-PEG-COOH (5 kDa) was added (193 μl, 50 mg/ml) to the partially coated GNPs to coat 20% of particle surface. The solution was then mixed for three hours at 4° C. followed by centrifugation at 14,000 g for 30 minutes at 4° C. Insulin was then covalently conjugated to the free carboxylic groups of the HS-PEG-COOH (5 kDa) by addition of excess amount of insulin together with EDC and NHS. The solution was then stirred for 3 hours at 4° C. followed by additional centrifugation at 14,000 g for 30 minutes at 4° C.

Conjugation of PEG3500 and anti-IgG1 to the GNPs: In order to further conjugate IgG1 antibodies to the GNPs, 135 μl of HS-PEG-COOH (˜3.5 kDa) solution (50 mg/ml) were added to the partially coated GNPs to coat the remaining 20% of particle surface. The solution was then mixed for two hours at 4° C. followed by centrifugation at 14,000 RPM for 30 minutes. Then, anti-IgG1 was covalently conjugated to the free carboxylic groups of the HS-PEG-COOH (˜3.5 kDa) by addition of 135 μg of anti-IgG1 antibody together with EDC and NHS. The solution was then stirred for 2 hours at 4° C. followed by centrifugation to remove unbound antibodies, until a final concentration of 25 mg/ml Au was reached.

For control experiments, GNPs conjugated with insulin and either anti-IgG1 or cisplatin were prepared (i.e., IgG1&Ins-GNPs and cisplatin& Ins-GNPs).

Delivery of the cisPt&IgG1&Ins-GNPs Conjugated GNPs into Mice Brain

Male BALB/c mice were divided into four treatment groups:
Group 1: Anti-IgG1 & insulin-conjugated GNPs (IgG1&Ins-GNPs; n=2)
Group 2: Cisplatin & insulin-conjugated GNPs (CisPt&Ins-GNPs; n=2)
Group 3: Multifuctional GNPs (cisPt&IgG1&Ins-GNPs; n=2)
Group 4: Free cisplatin (n=2)

Mice were injected intravenously with IgG1&Ins-GNPs (200 μl, 25 mg/ml), cisPt&Ins-GNPs (200 μl of 25 mg/ml), cisPt&IgG1&Ins-GNPs (200 μl of 25 mg/ml), or with equivalent amounts of free cisplatin. Eight hours post injection, mice underwent perfusion to remove all the particles residing within the blood vessels. Then, mice brains were extracted and analyzed using ICP-OES to measure the amounts of Au and Pt that penetrated into the brain (FIGS. 6A and 6B, respectively).

As can be seen in FIG. 5A and FIG. 5B, following IV injection of cisPt&IgG1&Ins-GNPs, significant amounts of both gold and platin were found in mice brains indicating successful penetration of the multifunctional GNP through the BBB. Remarkably, FIG. 6B shows that the amount of Pt found within the brain of mice administered with either cisPt&Ins-GNPs or cisPt&IgG1&Ins-GNPs was significantly higher than that after administration of equivalent dose of free cisplatin, indicating that the multifunctional GNP platform enhances the penetration of the small molecule cisplatin through the BBB.

### Example 5: The Effect of Linkers Length on the Ability of the Nano Delivery System to Cross the BBB

Several types of gold nanoparticles conjugated to insulin, IgG1 antibody and Iba1 antibody are synthesized as described in Example 1, with different combinations of PEG linkers, as specified in Table 1.

TABLE 1
Dual antibody GNPs synthesized with different linkers' lengths.

| | 1^{st} polymeric linker | | 2^{nd} polymeric linker | | 3^{rd} polymeric linker | | | |
|---|---|---|---|---|---|---|---|---|
| | (HS-PEG-COOH | | (HS-PEG-COOH | | (HS-PEG-COOH | | | |
| GNP | conjugated to a | | conjugated to a | | conjugated to | | mPEG-SH | |
| # | first Antibody) | | second antibody) | | insulin) | | as capping | |
| 1 | ~3.5 | kDa | ~3.5 | kDa | ~5 | kDa | ~5 | kDa |
| 2 | ~5 | kDa | ~5 | kDa | ~3.5 | kDa | ~3.5 | kDa |
| 3 | ~5 | kDa | ~5 | kDa | ~1 | kDa | ~5 | kDa |
| 4 | ~3.5 | kDa | ~3.5 | kDa | ~3.5 | kDa | ~3.5 | kDa |
| 5 | ~1 | kDa | ~1 | kDa | ~1 | kDa | ~1 | kDa |
| 6 | ~5 | kDa | ~5 | kDa | ~5 | kDa | ~5 | kDa |

In order to examine the effect of linkers' lengths on the ability of the nano delivery system to cross the BBB, the brain-targeted particles listed in Table 1 are intravenously injected (200 μl of 30 mg/ml) into the tail vein of male Balb/C mice. 8 hrs post injection, mice are sacrificed and undergo perfusion. Then, the brains are extracted and analyzed by ICP-OES or by ICP-MS to quantify the amount of gold that penetrated through the BBB.

Interestingly, the highest brain penetration is observed with GNPs in which the first and second polymeric linkers are shorter than the third polymeric linker. It is hypothesized that in order to achieve an efficient penetration through the BBB into the brain, the insulin which acts as the brain internalizing moiety should be exposed on the surface of the entire nano-delivery system (i.e., to be present on the external surface of the particle). Since insulin is significantly smaller than the antibodies (5 kDa compared to approximately 150 kDa), it has to be conjugated to a linker which is longer than the linker used to bind the antibody, in order to remain exposed on the nano-delivery system surface and not to be shielded by the antibody.

### Example 6: The Effect of Dual-Antibody GNPs on Cancer Cells: An In Vitro Study

GNPs conjugated to insulin and two different anti-HER2 antibodies (Trastuzumab and Pertuzumab; Brockhoff et al., Cell Prolif. 2007, 40, 488-507 and Scheuer et al., Cancer Res. 2009, 69(24), 9330-9336) are prepared according to the protocol described in Example 1. For comparison, GNPs conjugated to insulin and a single antibody (either Trastuzumab or Pertuzumab) are also prepared.

BT474, a HER-2 positive breast cancer cell line is used to determine the effect of GNPs conjugated to each of the antibodies as a single therapy and their combined effect. Cells are treated with Trastuzumab-GNPs, Pertuzumab-GNPs, or Trastuzumab&Pertuzumab-GNPs at different concentration. Untreated cells serve as control samples.

The effect of the various treatments on the cells is examined using cell cycle arrest determination, apoptosis and proliferation assays. Each treatment is performed in triplicates.

### Example 7: In Vivo Brain Efficacy of the Bi-Specific GNP Complex

The efficacy of the platform as a multi-functional drug carrier was tested. For this experiment, both Trastusumab and Pertusumab antibodies which are considered the 1st line of treatment for breast cancer tumors and are given together, were used. Both antibodies were conjugated to the GNPs and their efficacy was tested in a metastatic breast cancer brain tumor mice model. 150K breast carcinoma BT474 cells were inoculated into the brains of NOD-SCID mice with the following bregma coordinates: 0.5 Anterior, 1.7 lateral, 3.5 depth. The tumor growth was monitored through BLI imaging every week. Three weeks post tumor inoculation the mice were divided into 4 groups according to the tumor size as measured using MRI; a control group that received Saline, Control group that received the mixture of the free antibodies, and the test group that received the bi-functional GNPs (with both antibodies conjugated to the same particle). The treatment composition (40 mg/kg Abs) was injected IP once a week, for 4 consecutive weeks. At this point an MRI scan was conducted to measure again the tumor size. Results indicated that the bi-functional particles delayed tumor growth as compared to the untreated mice or the free antibodies treatment (FIG. 7a). The brains where then extracted and the particles penetration was confirmed through ICP-OES. FIG. 7b shows 3 representative brains, where the tumor is seen clearly within a brain on a mouse that received bi-functional GNPs, due to the purple color of the particles that accumulated within the tumor.

### Example 8: Multifunctional GNPs in Combination with Radiation Therapy for Cancer Treatment: An In Vivo Study

Gold nanoparticles conjugated to anti-EGFR (CTX) and Temozolomide (TMZ) are prepared according to the protocol in Example 2.

Mice (n=25) are divided into 5 treatment groups as indicated in Table 2 below (n=5 per group), in order to examine the contribution of different factors to the treatment outcome.

TABLE 2
Treatment groups in the in vivo study

| Group | Treatment type |
|---|---|
| 1 | Control group |
| 2 | Free TMZ + radiation (standard of care) |
| 3 | Free TMZ + radiation + free CTX |
| 4 | TMZ&CTX-GNPs |
| 5 | TMZ&CTX-GNPs + radiation |

Mice are injected intracranially with human U87 GBM cells (3*10\hat{ }4-3*10\hat{ }5); the injection site is 2 mm posterior and 1.5 mm lateral to the bregma. MRI imaging is used to verify tumor development which is measured at about 14 days after induction.

Mice in groups 2-3 receive standard treatment including intraperitoneal TMZ (10 mg/kg for 5 days). Intravenous CTX (1 mg/kg) is administered to the mice in groups 3; Mice in groups 4 and 5 are administered intravenously with TMZ&CTX-GNPs that contain equivalent amounts of TMZ and CTX (10 mg/kg and 1 mg/kg, respectively). Radiation is be performed fractionated 6 MV X-ray irradiation to the whole brain (10Gy in 5 days, 2Gy/day).

Mice are sacrificed when clinically deteriorating or at the end of study protocol about 180 days after tumor injection.

During the follow up period, mice survival and wellbeing is monitored.

After scarification, brains are analyzed with immunohistochemistry.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A multifunctional particle comprising:
(a) an inorganic particle bound to at least: (i) a first linear polymeric linker; (ii) a second linear polymeric linker; and (iii) a third linear polymeric linker;
(b) a first biologically active molecule conjugated to the first linear polymeric linker;
(c) a second biologically active molecule conjugated to the second linear polymeric linker; and
(d) a brain internalizing transporter moiety conjugated to the third linear polymeric linker,
wherein the length of the third linear polymeric linker is substantially different than the lengths of the first and the second linear polymeric linkers,
wherein the molecular weight of the third polymeric linker is different than the molecular weight of the first and the second polymeric linkers in at least about 1000 Da, and wherein the first biologically active molecule is distinct from the second biologically active molecule.

2. The multifunctional particle according to claim 1, wherein the length of the third linear polymeric linker is substantially higher than the lengths of the first and the second linear polymeric linkers.

3. The multifunctional particle according to claim 1 or claim 2, wherein at least one of the first, the second and the third polymeric linkers are non-cleavable under physiological conditions.

4. The multifunctional particle according to any one of claims 1 to 3, wherein the first, the second and the third polymeric linkers are non-cleavable under physiological conditions.

5. The multifunctional particle according to any one of claims 1 to 4, wherein the molecular weight of the first and the second linear polymeric linkers is within the range of 1,000-10,000 Da and wherein the molecular weight of the third linear polymeric linkers is within the range of 2,000-11,000 Da.

6. The multifunctional particle according to any one of claims 1 to 5, wherein the molecular weight of the third linear polymeric linker is higher than the molecular weights of the first and the second linear polymeric linkers.

7. The multifunctional particle according to any one of claims 1 to 6, wherein the third linear polymeric linker is composed of repeating monomer units and at least one of the first and the second linear polymeric linkers is composed of the same repeating monomer units as the third linear polymeric linker, and wherein the third linear polymeric linker has a different number of repeating monomer units than the at least one of the first and the second linear polymeric linkers.

8. The multifunctional particle according to any one of claims 1 to 7, wherein the first polymeric linker and the second polymeric linker are identical.

9. The multifunctional particle according to any one of claims 1 to 8, wherein the first and the second linear polymeric linkers are bound to the inorganic particle through a sulfide bond, and the first and the second biologically active molecules are conjugated to the respective linear polymeric linker through an amide bond.

10. The multifunctional particle according to any one of claims 1 to 9 wherein the first and the second biologically active molecules are independently selected from the group consisting of a polypeptide, an antibody, a peptide, a small molecule, an oligonucleotide, an antisense RNA, and any fragment or combination thereof.

11. The multifunctional particle according to claim 10, wherein the first and the second biologically active molecules are both an antibody or an antibody fragment thereof.

12. The multifunctional particle according to claim 10, wherein the first biologically active molecule is an antibody or a fragment thereof and the second biologically active molecule is a small molecule.

13. The multifunctional particle according to any one of claims 1 to 12, wherein the third linear polymeric linker constitutes from about 10% mol to 40% mol of the total polymeric linkers bound to the inorganic particle.

14. The multifunctional particle according to any one of claims 1 to 13, wherein each one of the first and the second linear polymeric linkers independently constitutes from about 5% mol to 40% mol of the total polymeric linkers bound to the inorganic particle.

15. The multifunctional particle according to any one of claims 1 to 14, wherein the first, the second and the third linear polymeric linkers independently comprise a polymer selected from the group consisting of: a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives, and combinations thereof.

16. The multifunctional particle according to claim 15, wherein at least one of the first, the second and the third linear polymeric linkers is a polyether, wherein the polyether is polyethylene glycol (PEG).

17. The multifunctional particle according to claim 16, wherein polyethylene glycol (PEG) is selected from a thiolated PEG acid (HS-PEG-COOH) and a thiolated PEG amine (HS-PEG-NH₂), wherein a thiolated end of the PEG is bound to the inorganic particle and an acid or amine end is conjugated to the brain-internalizing transporter moiety or to the respective biologically active molecule.

18. The multifunctional particle according to any one of claims 1 to 17, further comprising a fourth polymeric linker bound to the inorganic particle, wherein the fourth polymeric linker is monofunctional capping moiety.

19. The multifunctional particle according to claim 18, wherein said fourth polymeric linker comprises a polymer selected from the group consisting of a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives, and combinations thereof.

20. The multifunctional particle according to claim 19, wherein said fourth polymeric linker comprises a polyether, wherein the polyether is methoxy polyethylene glycol (mPEG).

21. The multifunctional particle according to any one of claims 1 to 20, wherein the inorganic particle is a nanoparticle selected from the group consisting of a metal nanoparticle, a metal oxide nanoparticle, a ceramic nanoparticle, and any combination thereof.

22. The multifunctional particle according to claim 21, wherein the nanoparticle comprises a metal selected from the group consisting of gold, silver, platinum, iron, and any combination thereof.

23. The multifunctional particle according to claim 21, wherein the nanoparticle comprises a metal oxide selected from the group consisting of iron oxide, magnesium oxide, nickel oxide, cobalt oxide, aluminum oxide, zinc oxide, copper oxide, manganese oxide, and any combination thereof.

24. The multifunctional particle according to any one of claims 21 to 23, wherein the inorganic nanoparticle is selected from the group consisting of a gold nanoparticle, an iron(III) oxide nanoparticle, and an iron(II,III) oxide nanoparticle.

25. The multifunctional particle according to any one of claims 1 to 24, wherein the brain-internalizing transporter moiety is selected from the group consisting of: insulin, an antibody specific for the insulin receptor, transferrin, an antibody specific for the transferrin receptor, a polypeptide that specifically binds to the transferrin receptor, a polypeptide that specifically binds to the insulin receptor, insulin-like growth factor 1, an antibody specific for the insulin-like growth factor receptor 1, a polypeptide that specifically binds to the insulin-like growth factor receptor 1, apolipoprotein A1, B, or E, lactoferrin, angiopep-2, a low-density lipoprotein, an antibody specific for low density lipoprotein receptor or lipoprotein receptor-related protein, a polypeptide that specifically binds to low density lipoprotein receptor or lipoprotein receptor-related protein, an antibody specific for diphtheria toxin receptor, a polypeptide that specifically binds to diphtheria toxin receptor, a BBB-penetrant cell-penetrating peptide (CPP), and any combination thereof.

26. The multifunctional particle according to claim 25, wherein the brain-internalizing transporter moiety is insulin or an analog, a derivative, a conjugate or a fragment thereof.

27. The multifunctional particle according to any one of claims 1 to 26, wherein the inorganic particle is a nanoparticle having a diameter of 10-160 nm.

28. The multifunctional particle according to any one of claims 1 to 27, further comprising a third biologically active molecule, wherein the third biologically active molecule is conjugated to a linear polymeric linker which is bound to the inorganic particle.

29. The multifunctional particle according to claim 28, wherein the third biologically active molecule is a chemotherapeutic molecule and wherein the linear polymeric linker is cleavable under physiological conditions.

30. A process for the preparation of the multifunctional particle according to any one of claims 1 to 29, the process comprising sequential steps of:
a) partially coating a surface of an inorganic particle with the first linear polymeric linker followed by conjugating said first linear polymeric linker to the first biologically active molecule;
b) partially coating the surface of the inorganic particle with the second linear polymeric linker followed by conjugating said second linear polymeric linker to the second biologically active molecule; and
c) partially coating the surface of the inorganic particle with the third linear polymeric linker followed by conjugating said third linear polymeric linker to the brain internalizing transporter moiety,
wherein steps (a), (b) and (c) can be performed in any order.

31. A process for the preparation of a multifunctional particle, the process comprising sequential steps of:
a) partially coating a surface of an inorganic particle with a first linear polymeric linker and a second linear polymeric linker, followed by conjugating the first and the second linear polymeric linkers to a first biologically active molecule and a second biologically active molecule, wherein the first linear polymeric linker and the second linear polymeric linker are identical and wherein the first biologically active molecule is distinct from the second biologically active molecule; and
b) partially coating the surface of the inorganic particle with a third linear polymeric linker followed by conjugating the third linear polymeric linker to a brain internalizing transporter moiety,
wherein the length of the third linear polymeric linker is substantially different than the length of the first and the second linear polymeric linkers, wherein the molecular weight of the third polymeric linker is different than the molecular weight of the first and the second polymeric linkers in at least about 1000 Da, and wherein step (a) and step (b) can be performed in any order.

32. The process according to claim 30 or claim 31, wherein the first polymeric linker has a first functional end group configured to bind the first biologically active molecule; the second polymeric linker has a second functional end group configured to bind the second biologically active molecule; and the third polymeric linker has a third functional end group configured to bind the brain internalizing transporter moiety, and wherein at least two of the first, the second and the third functional end groups are identical.

33. The process according to any one of claims 30 to 32, further comprising partially coating the surface of the inorganic particle with a fourth polymeric linker, wherein said fourth polymeric linker is a monofunctional capping moiety.

34. The process according to any one of claims 30 to 33, wherein each one of the first linear polymeric linker and the second linear polymeric linker is added in an amount suitable for covering between 5% and 40% of the surface of the inorganic particle, and the third linear polymeric linker is added in an amount suitable for covering between 5% and 40% of the surface of the inorganic particle.

35. A pharmaceutical composition comprising the multifunctional particle of any one of claims 1 to 29 and a pharmaceutically acceptable carrier.

36. The pharmaceutical composition of claim 35, being formulated for at least one of an intravenous (IV) administration, an intranasal (IN) administration, an intraperitoneal (IP) administration and an intrathecal (IT) administration.

37. The pharmaceutical composition of any one of claim 35 or 36, for use in the prevention, treatment, and/or monitoring of a brain-related disease or disorder in a subject in need thereof.

38. The pharmaceutical composition of claim 37, wherein the brain-related disease or disorder is a brain primary cancer or a secondary cancer.

39. A method for a simultaneous delivery of at least two biologically active molecules to a brain of a subject, the method comprising administering to the subject the pharmaceutical composition of any one of claim 35 or claim 36.

40. The method according to claim 39, wherein upon administration, the at least two biologically active molecules exhibit synchronized distribution within the brain.

41. A method for preventing, treating and/or monitoring a brain-related disease or disorder in a subject in need thereof, the method comprising administering to the subject the pharmaceutical composition of claim 35 or claim 36.

42. The method according to claim 41, further comprising a step of imaging a brain of the subject to thereby evaluate accumulation of the multifunctional particle in the brain of said subject.

43. The method according to claim 42, wherein the imaging is performed using an imaging system selected from the group consisting of: computed tomography imaging (CT), X-ray imaging, magnetic resonance imaging (MRI), positron emission tomography (PET), single-photon emission computed tomography (SPECT), ultrasound (US), and any combination thereof.

44. The method according to any one of claims 41 to 43, wherein the brain-related disease or disorder is a primary brain cancer or secondary brain cancer.

45. The method according to any one of claims 42 to 43, wherein the multifunctional particle is a radiosensitizer and wherein the method further comprises radiation therapy.

46. The method according to any one of claims 44 to 45, wherein the secondary brain cancer is selected from the group consisting of: breast cancer, lung cancer, melanoma, renal cancer and colorectal cancer.
